(19) United States
(12) Patent Application Publication
(10) Pub. No.: US 2026/0209871 A1
(43) Pub. Date: Jul. 23, 2026

(54) DIGITAL PCR ASSAY DESIGNS FOR MULTIPLE HEPATITIS B VIRUS GENE TARGETS AND NON-EXTENDABLE BLOCKER OLIGONUCLEOTIDES THEREFOR

(71) Applicant: Roche Molecular Systems, Inc., Pleasanton, CA (US)

(72) Inventors: Paul Joshua Dawson, Castro Valley, CA (US); Aaron Thaddeus Hamilton, Mountain House, CA (US); Calvin Mano, Berkeley, CA (US); Elizabeth Marie Scott, San Ramon, CA (US); Jingtao Sun, San Ramon, CA (US); Wei Yang, Foster City, CA (US)

(21) Appl. No.: 19/144,163

(22) PCT Filed: Dec. 22, 2023
(86) PCT No.: PCT/EP2023/087631

§ 371 Date: Jun. 27, 2025

Related U.S. Application Data
(60) Provisional application No. 63/435,798, filed on Dec. 28, 2022.

(51) Int. Cl. C12Q 1/70; C12Q 1/6806; C12Q 1/6825; C12Q 1/6848; C12Q 1/686

(52) U.S. Cl. CPC C12Q 1/706; C12Q 1/6806; C12Q 1/6825; C12Q 1/6848; C12Q 1/686

(57) ABSTRACT

The present invention provides compositions and methods relating to novel digital PCR (dPCR) assay designs (droplet digital or other systems) for the detection and quantitation of multiple hepatitis B virus (HBV) gene targets. The compositions and method can further include non-extendible blocker oligonucleotides for reduction of non-specific inter-amplicon extension.

### CROSS REFERENCE TO RELATED APPLICATIONS

This application is based on and claims priority from U.S. Provisional Patent Application No. 63/435,798, filed Dec. 28, 2022, which is incorporated by reference herein in its entirety.

### REFERENCE SEQUENCE LISTING

This application contains a Sequence Listing submitted as an electronic text file named “P38057-WO_Seq_Listing”, having a size in bytes of 75,424 bytes, and created on Dec. 19, 2023. The information contained in this electronic file is hereby incorporated by reference in its entirety pursuant to 37 CFR § 1.52(e)(5).

### FIELD OF THE INVENTION

The present disclosure relates to the field of in vitro viral diagnostics. Within this field, the present invention concerns the amplification and detection of a target nucleic acid that may be present in a sample and particularly, the specific amplification and detection of a target nucleic acid comprising sequence variations and/or individual mutations of Hepatitis B Virus (HBV), in particular, HBV RNA (in particular, HBV RNA derived from covalently-closed circular double-stranded DNA (cccDNA) such as HBV pre-genomic RNA (pgRNA)) as well as other HBV gene targets, optionally using at least one competitive blocking oligonucleotide for reduction of non-specific inter-amplicon extension. The invention further provides methods of, reaction mixtures for, and kits containing oligonucleotides (such as a reverse transcription (RT) primer and competitive blocking oligonucleotides) for the amplification and detection of various HBV RNA forms.

### BACKGROUND OF THE INVENTION

Hepatitis B is an infectious disease of the liver caused by HBV. Of note, HBV can cause both acute and/or chronic infections. During initial infection, many people are asymptomatic, whereas some develop rapid onset of sickness (including vomiting, yellowish skin, tiredness, dark urine and abdominal pain). Chronic hepatitis B preferentially afflicts those infected around the time of birth. Most of those individuals with chronic disease are also asymptomatic, but may eventually develop cirrhosis and liver cancer. These complications result in the death of 15% to 25% of those with chronic disease. In general, HBV is transmitted by exposure to infectious blood or body fluids, for example, when blood, semen, or another body fluid from a person infected with HBV enters the body of someone who is not infected. This can happen through sexual contact; sharing needles, syringes, or other drug-injection equipment; or from mother to baby at birth. Infection around the time of birth or from contact with other people's blood during childhood is the most frequent method by which hepatitis B is acquired in areas where the disease is common. In areas where the disease is rare, intravenous drug use and sexual intercourse are the most frequent routes of infection. Other risk factors include working in healthcare, blood transfusions, dialysis, living with an infected person, travel in countries where the infection rate is high, and living in an institution. An HBV infection can be diagnosed 30-60 days after exposure. The diagnosis is then usually confirmed by testing the blood for parts of the hepatitis B virus and for antibodies against HBV.

The 257 million chronic Hepatitis B infections remain a major health burden worldwide. There are therapies available to manage the disease; however, the cure rate is low. In the absence of a curative therapy, lifelong adherence to antiviral medication is required. Removal of the therapy often allows a rebound in HBV viral titer due to the inability of the current therapies to directly target the reservoir of HBV episomal genomes in the infected cell nuclei.

The virus life cycle for HBV alternates between DNA and RNA forms. The infectious HBV particle contains a relaxed-circle, incompletely double-stranded DNA genome (rcDNA). In an infected cell, the HBV DNA replication is completed to form a cccDNA in the nucleus of the host cell. Transcription from this DNA genome generates a variety of messenger RNA forms, which code for the proteins in the structure of the virus (core and surface proteins), the e antigen, the viral polymerase and the X antigen. One mRNA form called the pgRNA also serves as the template for the RT activity of the viral polymerase, which produces new copies of the rcDNA in encapsidated, secreted viral particles. There is also evidence that some proportion of encapsidated pgRNA is released without being reverse-transcribed, so that the production of an infected cell includes both rcDNA- and pgRNA-containing viral particles. In addition, there are multiple spliced RNA variants, some of which are also reverse transcribed into incomplete forms of HBV DNA and secreted. Integration of the HBV genome into a host chromosome is not a part of the replication cycle, as this cannot produce complete pgRNA molecules; however, it is a common occurrence and can result in host cells that produce smaller, truncated or fusion mRNAs, which contribute to the secretion of surface antigen containing subviral particles.

Below, in Table 1, is list of the HBV RNA forms that are believed to be generated from cccDNA of HBV.

TABLE 1
Type/Description

3.5 kb pgRNA, which is also the mRNA for core and polymerase proteins
3.5 kb precore mRNA, which is longer than pgRNA at 5′ end and
produces hepatitis B e antigen (HBeAg)
2.4 kb mRNA, large (preS1) surface proteins
2.1 kb mRNA, two transcription start sites for middle (preS2) and
small (HBs) surface proteins
0.7 kb mRNA, HBx regulatory protein
Shorter X-gene transcripts (later transcription start sites/5′ ends)
Truncated HBV mRNAs from a secondary 3′ end site in the X gene
(upstream of the primary poly(A) site) might be transcribed from cccDNA
but may be predominantly not from cccDNA
Spliced mRNAs. Spliced pgRNA can produce encapsidated spliced DNA.
Conserved major spliced forms; minor splice types may vary by genotype
Possibly two antisense RNA transcripts
Novel transcription start sites (TSS) or splice forms may be discovered

Below, in Table 2, is a list of the forms of HBV RNA that cannot be transcribed from integrated copies (i.e., are exclusively cccDNA in origin).

TABLE 2
Type/Description

3.5 kb pgRNA, which is also the mRNA for core and polymerase proteins
3.5 kb precore mRNA, which is longer than pgRNA at 5′ end and
produces HBeAg

Below, in Table 3, are some forms of HBV RNA that could be transcribed from integrated copies.

TABLE 3
Type/Description

Shorter mRNAs (not pgRNA or precore mRNAs) - depending on the
cut site in the genome of the integrated HBV copy, the integrated copy
may or may not be capable of producing one or more of the shorter
mRNAs (such as the 2.4 kb, 2.1 kb, 0.7 kb or other variants described)
Truncated HBV mRNAs from integrated HBV copies from a secondary
3′ end site in the X gene (upstream of the primary poly(A) site) -
the primary poly(A) site used in cccDNA may not be intact or used in
integrated copies
Fusion RNA products of HBV and host sequences from integrated HBV
copies (variable structures depending on integration site with the
human genome and promoter availability - it is possible either the
5′ end or 3′ end of such a transcript may contain human
sequence)

Markers for HBV include the detection of DNA, e antigen (from the precore mRNA), core antigen (or combinations of antigens including e and core), and s antigen, as well as the subject's or patient's production of antibodies for these antigens. Suppression of the s antigen is the marker for a functional cure. However, the s antigen can be produced by integrated, non-replicating copies of HBV and therefore quantitation of hepatitis B surface antigen (HBsAg) levels are unlikely to accurately reflect the pool of transcriptionally active cccDNA. DNA titer is monitored as a sensitive test to detect HBV infection and the decline in HBV is an indicator of treatment response. However, the current nucleoside analog therapies for HBV (which suppress reverse transcription) do not affect the transcription of pgRNA or other mRNAs, only the generation of new rcDNA copies. The decrease in DNA titers in the patient's blood (plasma or serum sample types) do not always correspond with the decline of HBV RNA, which may lag or even temporarily increase as encapsidated pgRNA (and spliced RNA) can be secreted by infected cells retaining transcriptionally active cccDNA. Because of this, HBV RNA has been explored as a separate marker for monitoring HBV disease state and therapy effectiveness. Studies have shown that HBV RNA levels can be predictive of outcomes such as e antigen loss, viral relapse, or “flare” events after the discontinuation of treatment, and the biomarker is potentially critical in timing the end of treatment for HBV patients.

Discrimination between HBV RNA forms is important to understanding the disease state and interpretation of molecular test results. Multiple mRNAs may be in circulation aside from the pgRNA (see, e.g., Stadelmayer et al., J Hepatol. 2020 vol. 73 pp. 40-51). These can vary in their transcription start sites but are overlapping due to the compact arrangement of the HBV genome (see, e.g., Altinel et al., J Virol. 2016 Nov. 14; 90(23):10811-10822). Most proposed HBV RNA detection assays have only one or two targets. The state-of-art assays target the poly(A) tail with a two-stage (RT and PCR) RACE method (see, e.g., van Bommel et al, Hepatology 2015 61:66-76; Zhang W et al, Methods Mol Med 2004, Vol 95 p 29-44.; Kairat A et al, Intervirology 1999, Vol 42, p 228-237). The poly(A) tail assays would detect pgRNA and other mRNAs that all end at the primary polyadenylation site (‘full length’ poly(A) tail). These assays would also detect spliced forms. However, to distinguish the pgRNA or each individual mRNA from other forms which are overlapping, a subtractive quantitation strategy would be needed requiring multiple PCR targets. For instance, the pgRNA and the precore mRNA (a slightly longer 3.5 kb mRNA) could be distinguished by targeting the region of the genome between the transcription start sites of these forms (see, e.g., Wang Jie et al, Journal of Hepatology 2016 V 65:700-710). Similar targeting of the 5′ end length differences of the mRNAs can also be used to discriminate between the pgRNA and the smaller viral mRNAs that produce the s antigen and X antigen (see, e.g., Butler E. K. et al., Hepatology. 2018 68(6):2106-2117). The X gene mRNA transcripts are known to be in circulation (see, e.g., Stadelmayer et al., J Hepatology 2020 vol. 73 pp. 40-51), and targets in this region would pick up both this transcript and longer mRNAs and pgRNA.

Other assays can target the spliced RNA variants which may be indicators of interferon therapy response (see, e.g., Chen et al., Sci Rep 5, 16459 (2015); Bayliss, J. et al., J Hepatol, 2013, V59, p 1022-1028, Preiss, S. et al., Hepatology, 2008, V48, 741-749); targets in the core region may be disrupted by splice variants, and so the proportion of these transcripts in a sample is relevant to accurate quantitation. The integrated HBV copies (which can be nearly-intact or fragmentary in different infected cells) cannot produce active virus but can generate s antigen producing transcripts, which interfere with human immune response and also reduce the predictive capability of s antigen monitoring as a marker for HBV viral production. The truncated RNAs from integrated copies of HBV, which end at a secondary poly(A) site upstream of the full-length site (van Bommel et al., Hepatology 2015 61:66-76), would not be picked up by a full-length 3′ end assay but may be detected by other targets within the S gene.

In light of the above, there is a need for assays having improved effectiveness with respect to distinguishing different HBV RNA forms.

### SUMMARY OF THE INVENTION

The present disclosure overcomes the aforementioned challenges by providing assays with improved effectiveness with respect to distinguishing different HBV RNA forms. In one aspect, the disclosure provides for a panel of multiple targeted assays to different HBV gene targets, all on the same platform, and capable of being multiplexed to reduce run to run variation.

Certain aspects in the present disclosure relate to methods for the rapid detection of the presence or absence of HBV RNA in a biological or non-biological sample, for monitoring HBV disease state and therapeutic efficacy, for example, detection of HBV by a polymerase chain reaction (PCR) in a single test tube. Such aspects include methods of detection of HBV comprising performing at least one cycling step, which may include an amplifying step and a hybridizing step. Furthermore, aspects include oligonucleotides (including a reverse transcription primer (which can also be a PCR primer), blocking oligonucleotide, conventional primers and probes), and kits that are designed for the detection of HBV in a single tube.

One sequence difference between HBV DNA and RNA is the poly(A) tail of the pgRNA and other mRNAs; however, methods that use oligo d (T) primers can detect non-target RNAs or other sequences with poly(A) stretches. “Anchored” poly(T)-containing oligonucleotides can provide some measure of specificity against non-target binding and extension however this is a tradeoff strategy which will result in some binding to the HBV DNA. The methods disclosed herein may include a competitive blocking oligonucleotide matching the DNA sequence at a target where the RNA sequence has a poly(A) tail junction, as a method for improving the performance (sensitivity and specificity) of an assay targeting RNA in the presence of DNA. The binding of the competitive blocking oligonucleotide to the homologous genomic HBV DNA prevents binding of the primer (e.g., RT primer), and therefore reduces the unwanted amplification of the homologous genomic HBV DNA. Modified stabilizing bases can be incorporated into the assay oligonucleotides or blocker oligonucleotides in order to further improve the discrimination capabilities of the method. Primers and probes can be provided that target the poly(A) tail of HBV RNA (in particular, HBV RNA transcribed from cccDNA, which has a standard poly(A) tail position for transcripts, such as pgRNA but also including other mRNAs and spliced RNAs). Competitive blocking oligonucleotides can be provided that increase specificity for RNA in the presence of HBV DNA. Additional primers and probes can be provided for that target other poly(A) sites, such as the secondary or truncated poly(A) site for HBV transcripts that can originate from integrated HBV copies. Competitive blocking oligonucleotides can be provided that increase specificity for RNA with these specific poly(A) sites in the presence of the homologous DNA.

One aspect of the present invention relates to a method to detect and quantify between two and six different Hepatitis B Virus (HBV) target nucleic acids in a sample by Digital PCR (dPCR), comprising, providing the sample, randomly distributing the sample into a plurality of equal sized and independent partitions, performing in each partition a dPCR assay with a plurality of forward and reverse primer sets and a plurality of probes, each probe labeled with a fluorescent dye that generates a signal that is different, for amplifying and detecting each of the HBV target nucleic acids, and measuring the amount of signal generated in each of the partitions to calculate the quantity of each HBV target nucleic acid in the sample. In one embodiment, the HBV target nucleic acids are selected from the group consisting of: Precore-mRNA 5′ end (non-pgRNA), Core, X gene, Truncated RNA 3′ end (poly(A) junction), Precore/core, Full-length RNA 3′ end (poly(A) junction), Selected splice junctions, S gene (pre splice site), S gene (post splice site) and pgRNA 5′ end. In another embodiment, the dPCR assay is performed using the forward and reverse primer sets and probes that are specific for the HBV target nucleic acid that are selected from the oligonucleotides listed in Tables 4 and 6. In one embodiment, the fluorescent dyes on the probe are selected from the group consisting of Atto-425, FAM, HEX, Texas Red, Cy5, and Cy5.5. In one embodiment, the method further comprises reducing unwanted amplification in the dPCR assay by the use of at least one blocker oligonucleotide having a non-extendable 3′ terminus and higher melting temperature (Tm) to template nucleic acid relative to the at least two forward and reverse primer sets and the at least two probes used in the dPCR assay. In some embodiments, the unwanted amplification may result from the amplification target regions being closely located. In another embodiment, the unwanted amplification is the presence of DNA template where RNA is the target nucleic acid. In yet another embodiment, the unwanted amplification is the presence of RNA splice variants in situations where the spliced RNA is the target and the unspliced RNA is inhibited.

In another aspect, a method to detect and quantify at least two different Hepatitis B Virus (HBV) target nucleic acids in a sample by Polymerase Chain Reaction (PCR) is provided, comprising providing the sample; randomly distributing the sample into a plurality of equal sized and independent partitions; performing in each partition a PCR assay with at least two forward and reverse primer sets for amplifying each of the HBV target nucleic acids, and with at least two probes, each probe labeled with a fluorescent dye that generates a different signal, for detecting each of the HBV target nucleic acids; and measuring the amount of signal generated in each of the partitions to calculate the quantity of each of the at least two different HBV target nucleic in the sample. In some embodiments, the PCR assay performed in each partition is a digital PCR (dPCR) assay. In some embodiments, the at least two different HBV target nucleic acids are selected from the group consisting of Precore-mRNA 5′ end (non-pregenomic RNA), Core, X gene, Truncated RNA 3′ end (poly(A) junction), Precore/core, Full-length RNA 3′ end (poly(A) junction), Selected splice junctions, S gene (pre splice site), S gene (post splice site) and pregenomic RNA 5′ end. In certain embodiments, the dPCR assay is performed using the forward and reverse primer sets and probes that are specific for the HBV target nucleic acid that are selected from the oligonucleotides listed in Tables 4 and 6. In some embodiments, the fluorescent dye on the labeled probe is selected from the group consisting of Atto-425, FAM, HEX, Texas Red, Cy5, and Cy5.5. In some embodiments, the method further comprises reducing unwanted amplification in the dPCR assay by the use of at least one blocker oligonucleotide having a non-extendable 3′ terminus and higher melting temperature (Tm) to template nucleic acid relative to the at least two forward and reverse primer sets and the at least two probes used in the dPCR assay. In some embodiments, the unwanted amplification may result from the amplification target regions being closely located. In another embodiment, the unwanted amplification is the presence of DNA template where RNA is the target nucleic acid. In yet another embodiment, the unwanted amplification is the presence of RNA splice variants in situations where the spliced RNA is the target and the unspliced RNA is inhibited.

Another aspect of the invention relates to a method of selectively detecting at least two targets in a sample, the method comprising performing an amplifying step comprising contacting the sample with a first set of primers to produce a first amplification product if a nucleic acid is present in the sample, a second set of primers to produce a second amplification product if the nucleic acid is present in the sample, and a blocker oligonucleotide; performing a hybridizing step comprising contacting the first and second amplification products with at least a first detectable probe and a second detectable probe; and detecting the presence or absence of the first and second amplification products, wherein the presence of the first amplification product is indicative of the presence of a first target in the sample and wherein the absence of the first amplification product is indicative of the absence of the first target in the sample, and wherein the presence of the second amplification product is indicative of the presence of a second target in the sample and wherein the absence of the second amplification product is indicative of the absence of the second target in the sample, and wherein the nucleic acid includes a continuous sequence comprising the first target, the second target, and an intermediate sequence positioned between the first target and the second target, and wherein the blocker oligonucleotide is complementary to at least a portion of the intermediate sequence. In some embodiments, the blocker oligonucleotide is not extendable by a DNA polymerase. In some embodiments, the blocker oligonucleotide reduces or eliminates the production of an unwanted amplification product comprising the first target and the second target. Another aspect of the present invention relates to a method of reducing unwanted amplification in a multiplex Digital PCR (dPCR) assay by the use of a blocker oligonucleotide having a non-extendable 3′ terminus and higher melting temperature (Tm) to template nucleic acid relative to the primers and probes used in dPCR. In one embodiment, the unwanted amplification results from the amplification target regions being closely located. In another embodiment, the unwanted amplification is the presence of DNA template where RNA is the target. In yet another embodiment, the unwanted amplification is the presence of RNA splice variants where the spliced RNA is the target and the unspliced RNA is inhibited.

In another aspect, a kit for selectively detecting at least two targets in a nucleic acid is provided, the kit comprising a first set of primers to produce a first amplification product if a first portion of a nucleic acid is present in the sample; a second set of primers to produce a second amplification product if a second portion of the nucleic acid is present in the sample; a blocker oligonucleotide complementary to the nucleic acid intermediate the first portion and the second portion; a first detectable probe complementary to the first amplification product; and a second detectable probe complementary to the second amplification product. In some embodiments, the blocker oligonucleotide is not extendable by a DNA polymerase. In some embodiments, the blocker oligonucleotide reduces or eliminates the production of an unwanted amplification product comprising the first target and the second target. Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting.

The details of one or more embodiments of the invention are set forth in the accompanying drawings and the description below. Other features, objects, and advantages of the invention will be apparent from the drawings and detailed description, and from the claims. All publications, patent applications, patents, and other references mentioned herein are incorporated by reference in their entirety. In case of conflict, the present specification, including definitions, will control.

### BRIEF DESCRIPTION OF THE DRAWINGS

FIG. 1 is a schematic illustration showing a diagram of the digital PCR assays disclosed herein on a linear schematic of the HBV RNA transcripts and selected splice variant forms. Elements (horizontal lines, boxes) of the full length HBV circular genome are illustrated along with a plurality of example primer pairs (pairs of arrows) for amplification of target regions of interest. Example primers are indicated for targets including precore mRNA 5′ end (non-pgRNA) 1, core target 2, X gene target 3, truncated RNA 3′ end (poly(A) junction) 4, precore/core target 5, full-length RNA 3′ end (poly(A) junction) 6, selected splice junctions (examples shown) 7, S gene, pre-splice site 8, S gene, post-splice site 9, and pgRNA+pc-mRNA 5′ end for 3.5 kb transcripts 10.

FIG. 2 is a schematic illustration showing the use of a blocker oligonucleotide to prevent unexpected amplifications as a result of two amplification regions located close to each other in a target template.

FIG. 3 is a schematic illustration showing the use of a blocker oligonucleotide binding to an intron sequence to inhibit DNA-specific amplification without affecting the RNA-specific amplification.

FIG. 4 shows the use of a blocker oligonucleotide designed to inhibit unspliced RNA variants. The example DNA template (top) is illustrated to show 3 exons and 2 introns associated with a single target gene.

FIG. 5 shows the use of a blocker oligonucleotide designed to inhibit a longer form of fusion RNA. The example DNA template (top) is illustrated to show two distinct regions with the first region (left) comprising 2 exons and 1 intron associated with a first target gene and the second region (right) comprising 1 exon associated with a second target gene.

FIG. 6 shows linearity plot for an HBV dPCR assay according to the present disclosure. Primers and probes were designed against six HBV targets, including i) 3′ precore, ii) 3′ poly(A), iii) X gene mRNA, iv) core gene mRNA, v) truncated poly(A) and vi) 5′-precore. The log of the measured concentrations for each of the six targets in the samples were calculated and plotted on the vertical axis against the known (expected) concentrations for those samples on the horizontal axis. Linear regressions for each series were calculated and plotted (dashed lines). Slope, intercept and R² values for each linear regression are shown in Table 5.

FIG. 7 shows a box and whisker plot for an HBV dPCR assay according to the present disclosure. Primers and probes were designed against six HBV targets, including i) 3′ precore, ii) 3′ poly(A), iii) X gene mRNA, iv) core gene mRNA, v) truncated poly(A) and vi) 5′-precore. The log of the measured concentrations for each of the six targets in the samples were calculated and plotted on the vertical axis against the known (expected) concentrations for those samples on the horizontal.

FIGS. 8A and 8B show a comparison of dPCR assay results for amplification of 3′ precore RNA (FIG. 8A) and 3′ precore DNA (FIG. 8B) using either a control reverse primer complementary to precore HBV (excluding the poly(A) tail) or an 8 bp poly(T) containing reverse primer complementary to precore HBV (including the poly(A) tail). Fluorescence signal as detected in channel 1 of the dPCR instrument is plotted on the vertical axis for each event (droplet detected) as shown on the lower horizontal axis. The well identifier for each sample (well ID) is shown on the upper horizontal axis, with data associated with each well ID delineated by (and contained between) vertical dashed lines.

FIG. 9 shows dPCR assay results for a plurality of HBV S gene assays, including one S gene(S) assay and three S-gene Post-Splice (SPS) Assays. Fluorescence signal as detected in channel 1 of the dPCR instrument is plotted on the vertical axis for each event (droplet detected) as shown on the lower horizontal axis. The well identifier (and associated assay) for each sample is shown on the upper horizontal axis, with data associated with each well delineated by (and contained between) vertical dashed lines.

FIG. 10 is a sensitivity plot illustrating measured titer (cps/μL) on the vertical axis as compared with the expected (input) concentration (log cps/μL) for Sps Assay 2 of FIG. 9. A strong correlation between input and measured concentration was determined by linear regression (R²=0.9991).

FIG. 11 is a schematic illustration of an HBV 3.5 kb RNA assay targeting the pre-splice site of core region for the detection of both pgRNA and precore mRNA. The target region of the assay is indicated by a dashed box.

FIG. 12 shows a detail view of the schematic illustration of FIG. 11. The target region of the assay is indicated by a dashed box. Two in vitro templates (IVT) comprising the target region (IVT 14 and IVT 15) are illustrated in alignment with the corresponding site on the HBV RNA.

FIG. 13 shows dPCR assay results for the pgRNA IVT assay illustrated in FIGS. 11 and 12. Fluorescence signal as detected in channel 1 of the dPCR instrument is plotted on the vertical axis for each event (droplet detected) as shown on the lower horizontal axis. The well identifier for each sample is shown on the upper horizontal axis, with data associated with each well delineated by (and contained between) vertical dashed lines.

FIG. 14 shows dPCR assay results for the IVT 14 template as detected in the assay illustrated in FIGS. 11 and 12. The assay demonstrates a high level of sensitivity, capable of detecting the target template (e.g., IVT 14) down to 10¹ cps/μL. Fluorescence signal as detected in channel 1 of the dPCR instrument is plotted on the vertical axis for each event (droplet detected) as shown on the lower horizontal axis. The well identifier (and associated input concentration) for each sample is shown on the upper horizontal axis, with data associated with each well delineated by (and contained between) vertical dashed lines. Saturated reactions (in which no negative droplets were detected) were observed for IVT 14 input concentrations of 10⁵ and 10⁶ cps/μL, whereas both positive and negative reaction droplets were observed for each of the other concentrations tested.

FIG. 15 is a linearity plot illustrating measured titer (cps/μL) on the vertical axis as compared with the expected (input) concentration (log cps/μL) for the 3.5 kb RNA Assay of FIG. 14. A strong correlation between input and measured concentration was determined by linear regression (R²=0.9992).

FIG. 16 is a linearity plot illustrating measured titer (cps/μL) on the vertical axis as compared with the expected (input) concentration (log cps/μL) for the IVT 14 template as detected in the 3.5 kb RNA Assay of FIG. 14. A strong correlation between input and measured concentration was determined by linear regression (R²=0.9999).

FIG. 17 shows dPCR assay results for a multiplex assays directed towards detection of core, X gene, and poly(A) targets in HBV RNA. The assay involves ratio-based multiplexing with core target 100% in channel 1, poly-A target 100% in channel 2, and X-gene target 50% in channel 1 and 50% in channel 2. Fluorescence signal as detected in either channel 1 or channel 2 of the dPCR instrument is plotted on the horizontal and vertical axis, respectively, for each event (droplet detected). Inter-cluster rain (corresponding to unexpected and/or undesirable amplification products) observed between clusters of data corresponding to each of the targets is denoted by rounded rectangular boxes.

FIG. 18 is a schematic illustration of an assay directed towards detection of core, X gene, and poly(A) targets in HBV RNA. Non-extendable blockers oligonucleotides were designed to bind to the indicated region between X gene and poly(A) to block the potential formation of hybrid amplicons including X gene and poly(A). In addition to the binding region for the blocker oligonucleotides, forward (Fwd) and reverse (Rev) primer pairs and the corresponding probes are indicated for each of the three targets.

FIG. 19 shows ddPCR assay results for a multiplex assay including non-extendable blockers oligonucleotides directed towards detection of core, X gene, and poly(A) targets in HBV RNA as shown in FIG. 18. The assay involves ratio-based multiplexing with core target 100% in channel 1, poly-A target 100% in channel 2, and X-gene target 50% in channel 1 and 50% in channel 2. Fluorescence signal as detected in either channel 1 or channel 2 of the dPCR instrument is plotted on the horizontal and vertical axis, respectively, for each event (droplet detected). Inter-cluster rain (corresponding to unexpected and/or undesirable amplification products) was significantly reduced or eliminated between clusters of data corresponding to each of the targets as compared with an assay excluding non-extendable blockers oligonucleotides as shown in FIG. 17.

FIG. 20 is a schematic illustration of an assay directed towards detection of truncated poly(A) targets in HBV RNA. Non-extendable blocker oligonucleotide TR3_DD was designed to impede the binding of the truncated poly(A) reverse primer to its corresponding target region in the HBV RNA template. In addition to blocker oligonucleotide TR3_DD, forward and reverse primers and corresponding probes are indicated.

FIG. 21 shows dPCR assay results for the assay illustrated in FIG. 20 both with and without the use of the illustrated non-extendable blocker oligonucleotide. Fluorescence signal as detected in channel 1 of the dPCR instrument is plotted on the vertical axis for each event (droplet detected) as shown on the lower horizontal axis. The well identifier (and associated assay condition—i.e., whether non-extendable blocker was present (+) or absent (−) from the reaction) for each sample is shown on the upper horizontal axis, with data associated with each well delineated by (and contained between) vertical dashed lines. Both positive and negative reaction droplets were observed for the each of the conditions tested.

### DETAILED DESCRIPTION OF THE INVENTION

Diagnosis of HBV infection by nucleic acid amplification provides a method for rapidly, accurately, reliably, specifically, and sensitively detecting and/or quantitating the viral infection. A digital PCR assay for detecting HBV gene targets (for example, HBV pgRNA and the smaller viral mRNAs) in the presence of homologous HBV DNA in a non-biological or biological samples is described herein. Primers (including RT primers), competitive blocking oligonucleotides, and probes for detecting and quantitating HBV are provided, as are articles of manufacture or kits containing such primers, competitive blocking oligonucleotides, and probes. The increased specificity and sensitivity of digital PCR (dPCR) for the quantitating various forms of HBV RNA compared to other PCR methods make feasible the implementation of this technology for routine diagnosis of HBV infections and therapeutic efficacy, in the clinical laboratory.

The assay designs for droplet digital PCR (ddPCR) and digital PCR include multiple targets including 5′ end and 3′ end structures; overlapping mRNA forms quantitation by targeting regions before and after the transcription start sites; splice junction targeted assays; and assays for integrated-copy transcripts. The assays can be used as RT-PCR assays after DNA removal (DNA removal not necessary for poly(A) targeted designs), and can also detect DNA forms such as the incomplete HBV genomes resulting from reverse-transcribed spliced RNAs or integrated copies of DNA released from infected cells. These assays will allow assessment of disease state and biological effects of antiviral therapy.

The digital PCR assays disclosed in the present invention include: (i) Poly A targeted assays for full length mRNA and pgRNA 3′ ends; (ii) X gene targeted assays; (iii) Core targeted assays; (iv) Precore assays that target near the 3′ end but omit the poly(A) tail junction; (v) 5′ end assays for the precore mRNA, a 3.5 kb transcript slightly longer than the pgRNA; (vi) Truncated assay targeting the secondary poly(A) start site; (vii) S gene assays, in locations upstream and downstream of a common splice junction; (viii) An assay for the pgRNA+precore-mRNA 5′ end to capture unspliced, 3.5 kb transcripts; and (ix) Assays for specific splice junctions.

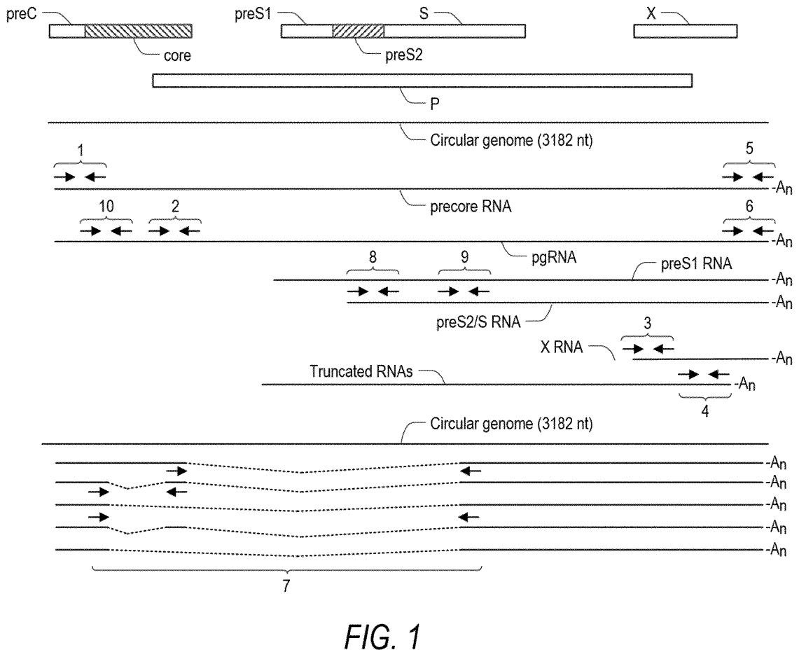

A diagram of the dPCR assays that presents a linear schematic of the HBV RNA transcripts and selected splice variant forms is shown in FIG. 1. These assays can be multiplexed in different combinations to save sample volume. Aside from the capabilities of the platform used for the PCR, the main restriction on multiplexing is the proximity or overlap of some of the target primer sets. The assay designs include novel oligonucleotide designs that are incorporated into the master mix as competitive, non-extensible blocking oligonucleotides that improves assay specificity for the targets when multiplexed. This blocker oligonucleotide is positioned between adjacent amplification products of the PCR assays, and reduces non-specific extension between the assay oligonucleotide sets across the regions between the intended targets. These assays are designed for digital PCR platforms, which may include droplet digital systems (such as the QX200 Droplet Digital PCR System from Bio-Rad) or systems with other forms of partitioning of reactions, including the Roche digital PCR system (Digital LightCycler®). More details of digital PCR and the use of blocking oligonucleotide designs to improve multiplexing are described below.

The present disclosure includes oligonucleotide primers (including RT primers), competitive blocking oligonucleotides, and fluorescent labeled hydrolysis probes that hybridize to the HBV nucleic acids, in particular HBV RNA (in particular, HBV RNA transcribed from cccDNA, such as pgRNA), in order to specifically identify and quantify various forms of HBV RNA.

The disclosed methods may include performing at least one cycling step that includes amplifying one or more portions of the nucleic acid molecule gene target from a sample using one or more pairs of primers. “HBV primer(s)” or “HBV RT primer(s)” as used herein refer to oligonucleotide primers that specifically anneal to nucleic acid sequences found in HBV or HBV RNA (such as HBV pgRNA), and initiate reverse transcription and/or DNA synthesis therefrom under appropriate conditions producing the respective amplification products. An example of a nucleic acid sequences found in HBV that is suitable for targeting include HBV pgRNA. Each of the discussed HBV primers (including RT primers) anneals to a target such that at least a portion of each amplification product contains nucleic acid sequence corresponding to the target. The one or more amplification products are produced provided that one or more nucleic acid is present in the sample, thus the presence of the one or more amplification products is indicative of the presence of HBV and/or HBV RNA (in particular, HBV RNA transcribed from cccDNA, such as pgRNA) in the sample. The amplification product should contain the nucleic acid sequences that are complementary to one or more detectable probes for HBV and/or HBV RNA. “HBV probe(s)” as used herein refer to oligonucleotide probes that specifically anneal to nucleic acid sequences found in the HBV target nucleic acid (e.g., HBV RNA). Each cycling step includes an amplification step, a hybridization step, and a detection step, in which the sample is contacted with the one or more detectable HBV or HBV RNA probes for detection of the presence or absence of HBV and/or HBV RNA (in particular, HBV RNA transcribed from cccDNA, such as pgRNA) in the sample. The term “blocking oligonucleotide” (or “competitive blocking oligonucleotide”, or “blocker”) as used herein refer to non-extensible oligonucleotides that specifically anneal to complement DNA and reduce the occurrence of non-specific inter-amplicon extension.

As used herein, the term “amplifying” refers to the process of synthesizing nucleic acid molecules that are complementary to one or both strands of a template nucleic acid molecule (e.g., nucleic acid molecules from the HBV and/or HBV RNA) Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” as used herein is known to those skilled in the art and refers to oligomeric compounds, primarily to oligonucleotides but also to modified oligonucleotides that are able to “prime” DNA synthesis by a template-dependent DNA polymerase, i.e., the 3′-end of the oligonucleotide provides a free 3′—OH group where further “nucleotides” may be attached by a template-dependent DNA polymerase establishing 3′ to 5′ phosphodiester linkage whereby deoxynucleoside triphosphates are used and whereby pyrophosphate is released. In some embodiments, the primer is also a reverse transcription (RT) primer (RT primer). There are several types of RT primers known in the art, including oligo(dT)N primers, anchored oligo(dT)N primers, random hexamer primers, and sequence specific primers. In some embodiments, the RT primer will anneal to RNA (e.g., HBV RNA), and extend to generate a DNA complement (i.e., reverse transcription of the target). In some embodiments, the RT primer targets poly(A)-containing HBV RNA, and therefore the RT primer is a poly-T containing oligonucleotide.

The term “hybridizing” refers to the annealing of one or more probes to an amplification product. “Hybridization conditions” typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished, if necessary.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a t-butyl benzyl, a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-0-methyl ribo-U, 2′-0-methyl ribo-C, an N4-ethyl-dC, an N6-methyl-dA, a 5-propynyl dU, a 5-propynyl dC, and N6 benzyl-dA, and the like. Some oligonucleotides described herein contain modified bases for increased stability or other improvements in performance. One example is 5-propynyl-dU (modified Uracil) which can replace a T (thymine). In oligonucleotide sequences provided the pdU, T, and U nucleotide designations would be considered interchangeable as the assays may contain either the modified or unmodified version of a particular oligonucleotide. Another example of a modified nucleotide includes locked nucleic acid (LNA). An LNA (also known as inaccessible RNA) is a modified RNA nucleotide in which the ribose moiety is modified with an extra bridge connecting the 2′ oxygen and the 4′ carbon. This bridge locks the ribose in the 3′-endo (North) confirmation, which is often found in the A-form duplexes. The effect of LNA is that the locked ribose conformation enhances base stacking and backbone pre-organization, which significantly increases the hybridization properties (melting temperature) of oligonucleotides. Many other modified nucleotides that can be substituted in the oligonucleotides are referred to herein or are otherwise known in the art. In certain embodiments, modified nucleotide substitutions modify melting temperatures (Tₘ) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference. Other modified nucleotide substitutions may alter the stability of the oligonucleotide, or provide other desirable features. For instance, some modifications can make an oligonucleotide non-extensible, which is useful for probes and for the competitive blocking oligonucleotides. Non-extensible ends can be facilitated by, in addition to a phosphate, a C3 spacer, a dideoxy nucleotide, attaching the 3′-end of a second oligonucleotide to the 3-end of an oligonucleotide, and the like.

Oligonucleotides including modified oligonucleotides and oligonucleotide analogs that amplify a nucleic acid molecule encoding the HBV target, e.g., nucleic acids encoding alternative portions of HBV can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In the assay, a “competitive blocking oligonucleotide,” “competitive blocking nucleotides,” “competitive blocking nucleic acids,” “blocking oligonucleotide,” “blocking nucleotides”, “blocker”, and/or “blocking nucleic acids” are employed, and are terms that refer to a competitive blocking oligonucleotides bind to a region in HBV DNA or RNA.

The set of forward primers for detection of the presence or absence of HBV nucleic acids, such as HBV RNA and other gene targets, include the sequences of SEQ ID NOs:20, 23, 24, 210, 213, 214, 387, and 389. The set of reverse transcription primers, which can function as reverse primers (e.g., RT/reverse primers) for detection of the presence or absence of HBV nucleic acids, such as HBV RNA (such as HBV derived from cccDNA, such as pgRNA), include the sequences of SEQ ID NOs:16, 18, 19, 25-30, 33-190, 206, 208, 209, 215-220, and 223-380. The set of competitive blocking oligonucleotides for increasing specificity of the detection of the presence or absence of HBV nucleic acids, such as HBV RNA (such as HBV derived from cccDNA, such as pgRNA), include the sequences of SEQ ID NOs:1-15, 21, 22, 191-205, 211, and 212. The set of probes for detection of the presence or absence of HBV nucleic acids, such as HBV RNA (such as HBV derived from cccDNA, such as pgRNA), include the sequences of SEQ ID NOs:17, 31, 32, 207, 221, 222, 381-386, 388, and 390-392.

In addition to a set of primers and competitive blocking oligonucleotides, the methods may use one or more probes in order to detect the presence or absence of HBV nucleic acid, such as HBV RNA (such as HBV derived from cccDNA, such as pgRNA). The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to HBV nucleic acids (including HBV RNA, such as HBV RNA transcribed from cccDNA, such as pgRNA) (target) nucleic acid. A “probe” can be referred to as a “detection probe” meaning that it detects the target nucleic acid.

In some embodiments, the described HBV nucleic acid probes (including probes for HBV RNA) can be labeled with at least one fluorescent label. In one embodiment, the HBV nucleic acids probes (including probes for HBV RNA) can be labeled with a donor fluorescent moiety, e.g., a fluorescent dye, and a corresponding acceptor moiety, e.g., a quencher. In one embodiment, the probe comprises or consists of a fluorescent moiety and the nucleic acid sequences comprise or consist of SEQ ID NOs:17, 31, 32, 207, 221, 222, 381-386, 388, and 390-392.

Designing oligonucleotides to be used as probes can be performed in a manner similar to the design of primers. Embodiments may use a single probe or a pair of probes for detection of the amplification product. Depending on the embodiment, the probe(s) used may comprise at least one label and/or at least one quencher moiety. As with the primers, the probes usually have melting temperatures appropriate for the thermal cycling parameters of the amplification method, and the length of each probe must be sufficient for sequence-specific hybridization to occur but not so long that fidelity is reduced during synthesis. Oligonucleotide probes are generally 15 to 40 (e.g., 16, 18, 20, 21, 22, 23, 24, or 25) nucleotides in length.

Constructs can include vectors each containing one or more of the sequences of the primers, competitive blocking oligonucleotides, and probes nucleic acid molecules for HBV (e.g., SEQ ID NOs:1-392). Constructs can be used, for example, as control template nucleic acid molecules. Vectors suitable for use are commercially available and/or produced by recombinant nucleic acid technology methods routine in the art. HBV nucleic acid molecules can be obtained, for example, by chemical synthesis, direct cloning from HBV, or by nucleic acid amplification.

Constructs suitable for use in the methods typically include, in addition to the HBV nucleic acids molecules (e.g., a nucleic acid molecule that contains one or more sequences of SEQ ID NOs:1-392), sequences encoding a selectable marker (e.g., an antibiotic resistance gene) for selecting desired constructs and/or transformants, and an origin of replication. The choice of vector systems usually depends upon several factors, including, but not limited to, the choice of host cells, replication efficiency, selectability, inducibility, and the ease of recovery.

Constructs containing HBV nucleic acids molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

Constructs (plasmid vectors) can be used to generate RNA molecules, through an in-vitro transcription or other process, producing RNA templates which may also contain the binding sites of primers and probes. RNA template molecules can also be created by synthesis. A type of RNA template that can be created as a control material is an armored RNA (an RNA molecule that is enclosed within a protein coat), involving the production of RNA and a coat protein (such as a viral capsid protein) by a construct (for instance in a bacterial host) and assembly of the coat protein enclosing the RNA molecule. DNA molecules can also be enclosed in a protein coat for use as a control material.

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described HBV nucleic acid sequences (e.g., SEQ ID NOs:15, 18-20, 23-30, 33-190, 206, 208-210, 213-220, and 223-380). In some embodiments, the primers are reverse transcription (RT) primers (RT primers). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 seconds to 4 minutes (e.g., 1 minute to 2 minutes 30 seconds, or 1.5 minutes).

If the double-stranded template nucleic acid is denatured by heat, the reaction mixture is allowed to cool to a temperature that promotes annealing of each primer to its target sequence. The temperature for annealing is usually from about 35° C. to about 65° C. (e.g., about 40° C. to about 60° C.; about 45° C. to about 50° C.). Annealing times can be from about 10 seconds to about 1 minute (e.g., about 20 seconds to about 50 seconds; about 30 seconds to about 40 seconds). If necessary, the reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 seconds to about 5 minutes (e.g., about 30 seconds to about 4 minutes; about 1 minute to about 3 minutes; about 1 minute 30 seconds to about 2 minutes).

The genome of a retrovirus or RNA virus is comprised of a ribonucleic acid, i.e., RNA. HBV is a pararetrovirus, which is a non-retrovirus that still uses reverse transcription in its replication process, requiring RNA made by host enzyme for viral replication. In such case, the template nucleic acid, RNA, must first be transcribed into complementary DNA (cDNA) via the action of the enzyme reverse transcriptase. Reverse transcriptases use an RNA template and a short primer complementary to the 3′ end of the RNA to direct synthesis of the first strand cDNA, which can then be used directly as a template for polymerase chain reaction. For general preparation of RNA, primers can also be random, or assay/target-specific, depending on the method.

PCR assays can employ HBV nucleic acid such as RNA (such as HBV pgRNA) or DNA (cDNA). The template nucleic acid need not be purified; it may be a minor fraction of a complex mixture, such as HBV nucleic acid contained in human cells. HBV nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers (e.g., the forward primers comprising SEQ ID NOs: 20, 23, 24, 210, 213, 214, 387, and 389; and the RT/reverse primers comprising SEQ ID NOs: 16, 18, 19, 25-30, 33-190, 206, 208, 209, 215-220, and 223-380) are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly-synthesized strands form a double-stranded molecule that can be used in the succeeding steps of the reaction. The steps of strand separation, annealing, and elongation can be repeated as often as needed to produce the desired quantity of amplification products corresponding to the target HBV nucleic acid molecules (including HBV RNA, such as HBV pgRNA). The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety (e.g., FAM) and a corresponding quencher (e.g., BlackHole Quenchers™ (BHQ) (such as BHQ2)), which may or not be fluorescent, and which dissipates the transferred energy in a form other than light. When the probe is intact, energy transfer typically occurs between the donor and acceptor moieties such that fluorescent emission from the donor fluorescent moiety is quenched the acceptor moiety. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ) (such as BHQ2), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the HBV RNA target nucleic acid sequence (including HBV RNA, such as HBV RNA transcribed from cccDNA, such as pgRNA). Upon hybridization of the oligonucleotide probes to the amplification product nucleic acid at the appropriate positions, a FRET signal is generated. Hybridization temperatures can range from about 35° C. to about 65° C. for about 10 seconds to about 1 minute.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a xenon lamp, a fiber optic light source, or other high intensity light source appropriately filtered for excitation in the desired range.

As used herein with respect to donor and corresponding acceptor moieties “corresponding” refers to an acceptor fluorescent moiety or a dark quencher having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced there between.

Fluorescent donor and corresponding acceptor moieties are generally chosen for (a) high efficiency Foerster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, helium-cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide that contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next.

### Principles of Digital PCR

Digital PCR (dPCR), sometimes referred to as droplet digital PCR, is a PCR-based method for quantification of DNA or RNA targets. In digital PCR, a reaction mixture containing target nucleic acids, primers, probes and other reagents is randomly distributed into many thousands of equal-sized, independent partitions and undergoes end-point PCR. TaqMan hydrolysis probes are commonly used to detect the amplification of targets and fluorescent signal of each partition is measured at the end. Partitions with no target nucleic acids will have relatively low fluorescence and thus are negative, while partitions started with one or more target nucleic acid molecules will have high fluorescence and thus are positive. For each reaction, the proportion of negative partitions provides the basis for absolute quantification using Poisson statistics.

Methods involving dPCR provide a rather new approach to nucleic acid detection and quantification that offer an alternative method to conventional real-time quantitative PCR for absolute quantification of nucleic acids and rare allele detection. A dPCR assay works by partitioning a sample of nucleic acids into many individual, parallel PCR reactions; some of these reactions contain the target molecule (positive) while others do not (negative). Following PCR analysis, the fraction of negative reactions is used to generate an absolute count of the number of target molecules in the sample. One of the key advantages of dPCR over real-time PCR is its superior accuracy of quantification. This advantage relies on inherent properties of dPCR as quantification only requires correct counting of positive partitions and the knowledge of the theoretical partition volume (the count number is not very sensitive to PCR efficiency). A quantification standard is not required. This eliminates potential quantification errors caused by the standard itself.

As detailed above, the dPCR sample is partitioned so that individual nucleic acid molecules within the sample are localized and concentrated within many separate regions (reaction areas). The partitioning of the sample allows estimation of the number of nucleic acids by assuming that the molecule population follows the Poisson distribution. As a result, each part will contain a negative or positive reaction (“0” or “1”, respectively). After PCR amplification, nucleic acids may be quantified by counting the regions that contain PCR end-product positive reactions. In conventional quantitative PCR, the quantitation result may depend on the amplification efficiency of the PCR process. However, dPCR is not dependent on the number of amplification cycles to determine the initial sample amount, eliminating the reliance on uncertain exponential data to quantify target nucleic acids and therefore provides absolute quantification.

As a next step, dPCR is performed with the sample in each reaction area of an array of reaction areas. In dPCR, the nucleic acid in question is amplified and detected, where a number of individual molecules are each isolated in a separate reaction area. Each reaction area (well, chamber, bead, emulsion, etc.) will have either a negative result, if no starting molecule is present, or a positive result for amplification and detection, if the targeted starting molecule is present. It is a technique where a limiting dilution of the sample is made across a number of separate PCR reactions such that part of the reactions have no template molecules and give a negative amplification result. In counting the number of positive PCR reactions at the reaction endpoint, the individual template molecules present in the original sample one-by-one are counted. PCR-based techniques have the additional advantage of only counting molecules that can be amplified, e.g., that are relevant to the massively parallel PCR step in the sequencing workflow. In the digital PCR-based methods, one distributes the nucleic acid to be analyzed into a number of different reaction areas (such as well, beads, emulsions, gel spots, chambers in a microfluidic device, etc.). It is important that some reaction areas, but not all, contain at least one molecule. Typically, each reaction area will contain one or zero molecules. In practice, there will be a random distribution of molecules into reaction areas such as wells. In the case where a percentage of reaction areas (e.g., 80%) is positive, a number of areas will contain one or more molecules (e.g., an average of 2.2 molecules per well). Statistical methods may be used to calculate the expected total number of molecules in the sample, based on the number of different reaction areas and the number of positives. This will result in a calculated amount or concentration of nucleic acids in the portion that was applied to the different reaction areas. A number of statistical methods based on sampling and probability can be used to arrive at this concentration. An example of such an analysis is given in Dube et al., arXiv:0809.1460v2 “Computation of Maximal Resolution of Copy Number Variation on a Nanofluidic Device using Digital PCR (2008),” found at arxiv.org, citation arXiv:0809.1460v2 [q-bio.GN], first uploaded on 8 Sep. 2008. The publication provides a series of equations that may be used to estimate the concentration of molecules and statistical confidence interval based on the number of reaction areas used in a digital PCR array and the number of positive results. Another example of this type of calculation may be found in U.S. Patent Application US 2009/0239308 A1.

Usually, a Poisson distribution is used to predict the digital regime where only a single DNA amplicon will occur in a randomly discretized volume reactor to favor only one DNA amplicon of interest per reaction volume. In this way, the PCR amplified signal (e.g., a fluorescence) emitted by each reactor volume is the product of only one amplicon and is isolated from all other discrete reactor volumes. Quantification is then achieved by counting how many digital reactors emit an amplified fluorescent signal corresponding to an intercalating dye or a particular DNA polymerase probe sequence. Since each reactor volume is limited to no more than a single DNA strand in the digital regime, one can correctly assume that 100% of its amplified fluorescence signal comes from only that one DNA strand and corresponding primer and probe set. However, a very low-concentration regime is usually not favorable with respect to imprecision of result.

A number of methodologies for dPCR exist. For example, emulsion PCR has been used to prepare small beads with clonally amplified DNA—in essence, each bead contains one type of amplicon of dPCR. Fluorescent probe-based technologies, which can be performed on the PCR products “in situ” (i.e., in the same wells), are particularly well suited for this application. U.S. Pat. No. 6,440,705, contains a more detailed description of this amplification procedure. These amplifications may be carried out in an emulsion or gel, on a bead or in a multiwell plate. dPCR also includes microfluidic-based technologies where channels and pumps are used to deliver molecules to a number of reaction areas. Suitable microfluidic devices are known in the art.

The dPCR is carried out essentially as a conventional PCR. The nucleic acids (reference or of interest) in a suitable medium are contacted with primers, probes and a thermostable polymerase (e.g., Taq polymerase) and thermocycled (cycles of repeated heating and cooling of the reaction for separation of strands and enzymatic replication. The medium usually contains deoxynucleotides, a buffer solution and ions (e.g., Mg2+). The selectivity of PCR results from the use of primers that are complementary to the region targeted for amplification under specific thermal cycling conditions. The resulting amplification product is detected by use of a suitable probe, which is usually labelled, e.g., fluorescence-labelled. For mRNA-based PCR the RNA sample is first reverse-transcribed to complementary DNA (cDNA) with reverse transcriptase. Typically, the PCR process consists of a series of temperature changes that are repeated 25 to 50 times. These cycles normally consist of three stages: the first, at around 95° C., allows the separation of the nucleic acid's double chain; the second, at a temperature of around 50 to 60° C., allows the binding of the primers with the DNA template; the third, at between 68 to 72° C., facilitates the polymerization carried out by the DNA polymerase. Due to the small size of the fragments the last step is usually omitted in this type of PCR as the enzyme is able to increase their number during the change between the alignment stage and the denaturing stage. In addition, a signal, e.g., fluorescence, is measured with a temperature of, for example, 80° C., in order to reduce the signal caused by the presence of primer dimers when a non-specific dye is used. The temperatures and the timings used depend on a wide variety of parameters, such as: the enzyme used to synthesize the DNA, the concentration of divalent ions and deoxyribonucleotides (dNTPs) in the reaction and the binding temperature of the primers.

dPCR methods enable the unique ability to identify a greater number of fluorescent probe sequences (e.g., TaqMan probe sequences) by using multiple color, temporal, and intensity combinations to encode each unique probe sequence. Furthermore, less expensive non TaqMan-probe real-time PCR amplification indicators such as SYBR- or PicoGreen can be used to achieve multiplexed dPCR based on temporal cues alone, intensity cues alone, or intensity and temporal cues combined, thus distinguishing primer pairs at greater degrees with significant cost reductions. These can also be used to enhance controls and normalize results for greater accuracy if desired. The typical multiplexing limits from typical 5-plex qPCR can be increased to as much as 100-plex dPCR with limited spectral bands using fluorescent reporters.

There is a multitude of dPCR systems available, which may be used in the present invention. Commercialized digital PCR platforms include micro-well chip-based BioMark® dPCR from Fluidigm, through hole-based QuantStudio12 k flex dPCR and 3D dPCR from Life Technologies, and droplet-based ddPCR (ddPCR) QX100 and QX200 from Bio-Rad® and RainDrop from RainDance®. The microfluidic-chip-based dPCR can have up to several hundred reaction areas per panel. Droplet-based dPCR usually has approximately 20,000 partitioned droplets and can have up to 10,000,000 per reaction. The QuantStudio 12 k dPCR performs digital PCR analysis on an OpenArray® plate which contains 64 reaction areas per subarray and 48 subarrays in total, equating to a total of 3072 reaction areas per array.

Droplet dPCR (ddPCR) is based on water-oil emulsion droplet technology. A sample is fractionated into a multitude of droplets (e.g. about 20,000) and PCR amplification of the template molecules occurs in each individual droplet. ddPCR technology uses reagents and workflows similar to those used for most standard TaqMan probe-based assays including droplet formation chemistry. Also, an intercalating dye, such as Evagreen, may be used. The massive sample reaction partitioning is a key aspect of the ddPCR technique. Non-spherical partitions (e.g. nanowells) actually have a larger area per sample volume than the same number of spherical partitions.

Typically, the accuracy and more importantly the precision of determination by dPCR may be improved by using a greater number of reaction areas. One may use approximately, 100 to 200, 200 to 300, 300 to 400, 700 or more reaction areas, which are used for determining the amount or concentration in question by PCR. In a preferred embodiment of the methods of the present invention, the dPCR is carried out identically in at least 100 reaction areas, particularly at least 1,000 reaction areas, especially at least 5,000 reaction areas. In a preferred embodiment of the methods of the present invention, the dPCR is carried out identically in at least 10,000 reaction areas, particularly at least 50,000 reaction areas, especially at least 100,000 reaction areas.

Preferably, the dPCR involves the use of one or more fluorescent dPCR probes in order to detect one or more nucleic acid(s) of interest, particularly in combination with a quencher or as molecular beacon or as a hydrolysis probe. The dPCR may involve the use of one or more fluorescent probes in order to detect the nucleic acid of interest and/or the reference nucleic acid, particularly in combination with a quencher or as molecular beacon or as a hydrolysis probe. Representative donor and acceptor fluorescent moieties in FRET technology have been described above. To detect and quantify more than one target in the same reaction, primer and probe sets for each target can be combined for multiplexing. The Roche Digital LightCycler® dPCR system has six optical channels that allows for multiplexing of up to six targets in one reaction, using differently labeled TaqMan hydrolysis probes for each target. Other system with a lower number of optical channels may need more complicated strategies for multiplexing, e.g., use a combination of dyes for one probe.

### Use of Blocker Oligonucleotides for Reduction of Unexpected Amplifications

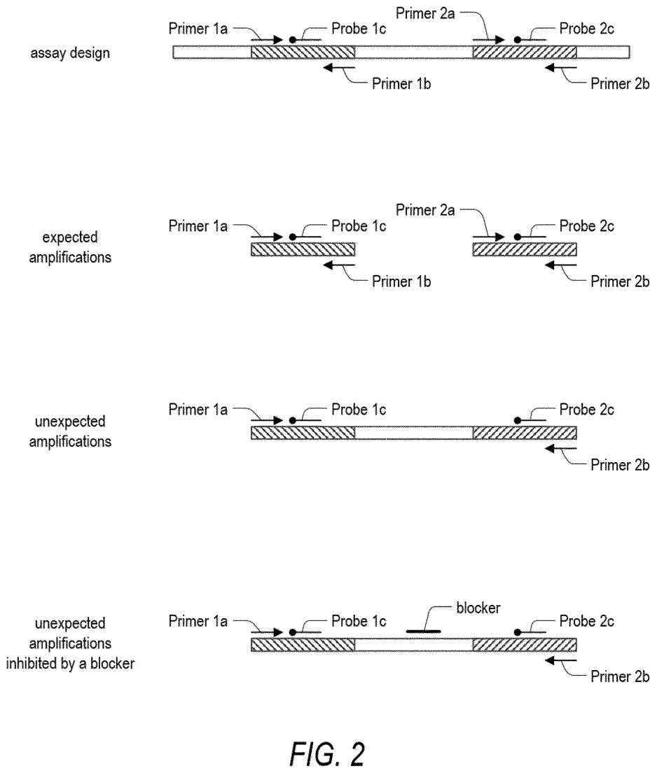

The biggest challenge in optimization of a multiplex assay is to make sure that there is no significant oligonucleotide interactions and assay interference. One possible case is that multiple primer and probe sets are overlapping in amplifiable sequences and thus creating multiple amplifications including the expected and unexpected ones. FIG. 2 shows examples in the HBV RNA assay design, where two amplifications are located close to each other in target template (within ~2 kb). Besides the expected amplifications that individual primer/probe sets were designed to, there might be additional unexpected amplification events from one primer of each assay and two probe cleaving events by one primer. These multiple amplifications and probe cleaving events may be at different efficiencies due to amplicon length and primer-probe distances, and thus end in a reduced end-point fluorescence of some partitions (“rain” phenotype) and even inaccurate quantification. A blocker oligonucleotide that is designed to the middle sequences, with a higher Tm (65° C.-90° C., enabled by Tm enhancer modification such as LNA) and non-extendable 3′-end (including 3-C spacer or phosphorylation), can effectively bind to the template with high affinity and inhibit the primer extension to the unwanted region. When a blocker oligonucleotide is added to the reaction, the “rain” phenotype in a dPCR reaction would be greatly reduced and the quantification result shall be improved.

General blocker oligonucleotide applications for non-HBV assays as well as HBV targets may include closely located amplification targets that are of interest of multiplexing. The two targets cannot share the same primer set because the resulting amplicon is outside of optimal size range of dPCR and may have sensitivity issues with sample fragmentation. This utility applies to both DNA and RNA assays (see FIG. 2).

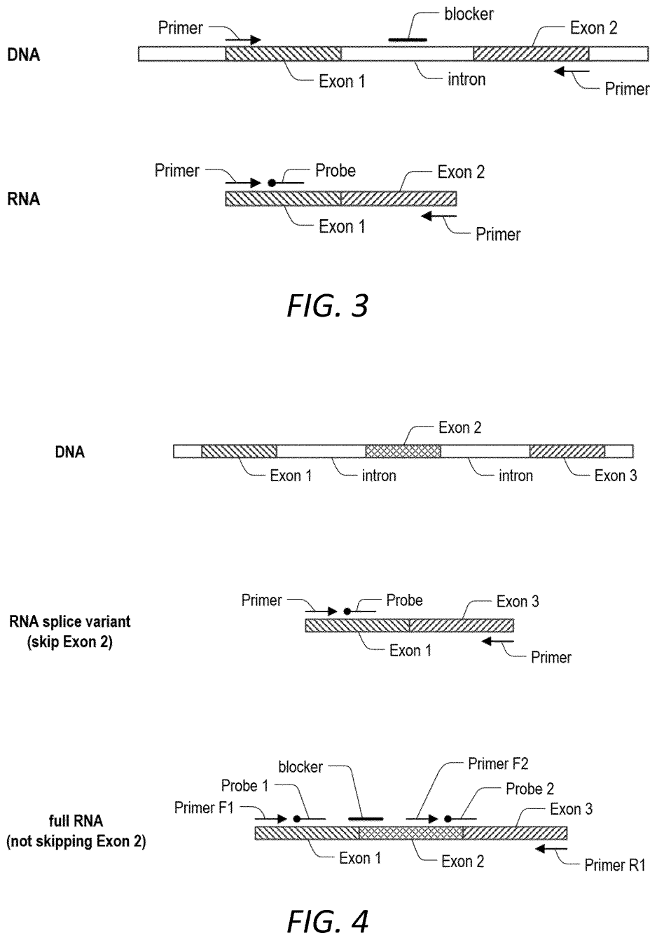

General blocker oligonucleotide applications for non-HBV assays as well as HBV targets may also include RNA assays that may cross-react with DNA template that is present in the sample. Even though the probe can be designed to the exon junctions to avoid cleaving from DNA amplification, it is still inevitable to deplete the primers and lower the on-target amplification efficiency. A blocker oligonucleotide can be designed to bind to the intron sequence that inhibit the DNA-specific amplification without affecting the RNA amplification (see FIG. 3)

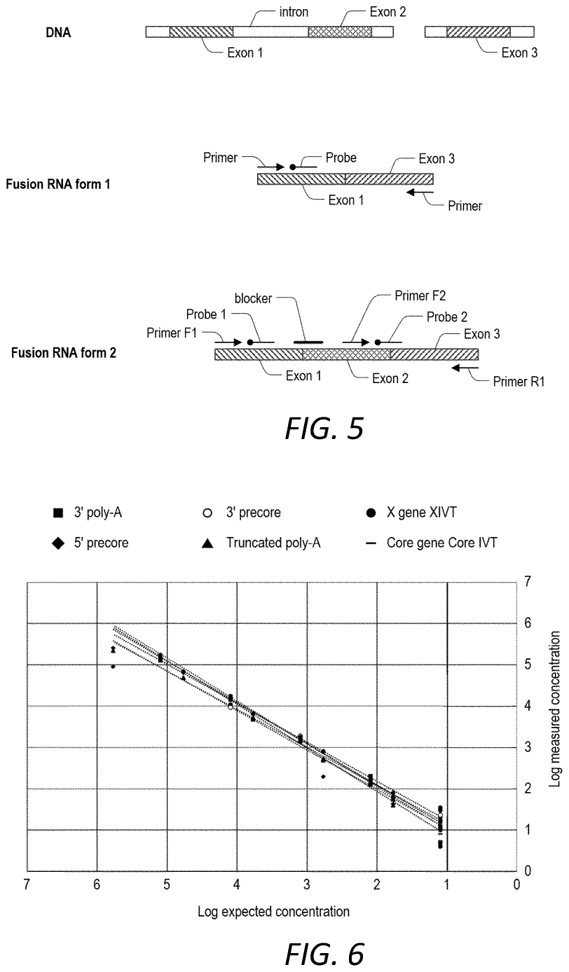

General blocker oligonucleotide applications for non-HBV assays as well as HBV targets may further include samples that are non-homogenous in template sequences. Such samples can include splice variants and fusion products. With respect to splice variants, some splice variant may generate a small amplicon that competes with the longer amplification from a non-spliced RNA species. In this case, a blocker oligonucleotide can be designed to inhibit the longer amplification and additional priming after the blocker may better multiplexed in this reaction (see FIG. 4). With respect to detection of multiple fusion products, when multiple fusion products have overlapping sequences, one set of primer pairs may generate amplicons with various length and thus different PCR efficiency. Long amplicon size over 300 bp is not optimal for digital PCR. In this case, a blocker oligonucleotide can be designed to inhibit the longer amplification. Additional priming after the blocker oligonucleotide may generate a compatible, similar-sized amplicon to the smaller fusion product, and could be discriminated using a second probe in different color (see FIG. 5).

### Articles of Manufacture/Kits

Embodiments of the present disclosure further provide for articles of manufacture or kits to detect HBV RNA and other gene targets. An article of manufacture can include primers and probes used to detect the HBV RNA target, together with suitable packaging materials. Representative primers and probes for detection of HBV RNA, including HBV RNA transcribed from cccDNA, such as HBV pgRNA are capable of hybridizing to HBV target nucleic acid molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to HBV target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the HBV probes (which may include probes that target HBV RNA). Examples of suitable FRET donor fluorescent moieties and corresponding acceptor fluorescent moieties are provided above.

Articles of manufacture can also contain a package insert or package label having instructions thereon for using the primers and probes to detect HBV (including HBV RNA), in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein. Embodiments of the present disclosure also provide for a set of primers and one or more detectable probes for the detection of HBV RNA, including HBV RNA in a sample. Additional primers and probes can be provided for that target other poly(A) sites, such as the secondary or truncated poly(A) site for HBV transcripts that can originate from integrated HBV copies.

Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention.

### Example 1

The present example illustrates sequences of oligonucleotides used in HBV dPCR assays. The nucleotide sequences and descriptions of the primer (forward and reverse), probe and blocking oligonucleotides used to perform the dPCR assays for the detection and quantitation of various HBV RNA forms and gene targets are listed in Table 4.

TABLE 4
3′ precore assays SEQ

| | | | SEQ |
|---|---|---|---|
| Target | Oligo Name | Sequence | ID NO: |
| 3′ precore | ESHBVPGRNA_ | GCAACTTTTTCACCTCTGCC | 1 |
| or poly(A) | DDPCR_FP3 | | |
| | ESHBVPGRNA_ | ATACGGGTCAATGTCCATGC | 2 |
| | DDPCR_RP3 | | |
| | ESHBV_HEX_PR3- | <5_HEX_ABD>TTCAAGCCT<ZEN>CCAAG | 3 |
| | Z_IBQ | CTGTGC<IB_FQ> | |
| | ESHBV_FAM_PR3- | <5_FAM_ABD>TTCAAGCCT<ZEN>CCAAG | 4 |
| | Z_IBQ | CTGTGC<IB_FQ> | |
| | HBV_PA | TTTTTTTTTTTTTTTTTTGAAGCTCC | 5 |
| Other | ESHBVPGRNA_ | ATACGGGTCAATGTCCATGT | 6 |
| | CDDPR_RP3_MIX | | |
| | ESHBVPGRNA_ | <FAM_Thr>TTCAAGCCTCCAAGCTGTGC | 7 |
| | DDPCR_PR3BHQ | <BHQ_2> | |
| | ESHBVPGRNA_ | <FAM_Thr>TTCAAGCCTCCAAGCTGTGC | 8 |
| | DDPCR_PR3_IBQ | <IB_FQ> | |

Core and X gene assays

| | | | SEQ |
|---|---|---|---|
| Target | Oligo Name | Sequence | ID NO: |
| Core | PGRNA- | GCGACGCGGCGATTGA | 9 |
| | C_R, 2419_2404 | | |
| | PGRNA- | GAGGCAGGTCCCCTAGAA | 10 |
| | C_F, 2355_2372 | | |
| | HC-SHORT- | <5_FAM_ABD>AGAACTCCC<ZEN>TCGCC | 11 |
| | 25_FAM_Z-IBFQ | TCGCAGACG<IB_FQ> | |
| | HC-SHORT- | <5_HEX_ABD>AGAACTCCC<ZEN>TCGCC | 12 |
| | 25_HEX_Z-IBFQ | TCGCAGACG<IB_FQ> | |
| | HC-LONG_HEX_Z- | <5_HEX_ABD>AGAACTCCC<ZEN>TCGCC | 13 |
| | IBFQ | TCGCAGACGAAG<IB_FQ> | |
| | HC-SHORT- | <5_HEX_ABD>AACTCCCTC<ZEN>GCCTC | 14 |
| | 23_HEX_Z-IBFQ | GCAGACG<IB_FQ> | |
| | PGRNA- | <HEX_Thr>AGAACTC<BHQ_2>CCTCGCCT | 15 |
| | C_HEX_7, 2375_2400 | CGCAGACGAAG<Phos> | |
| X gene | PGRNA- | GGTGAAGCGAAGTGCACA | 16 |
| | X_R, 1595_1578 | | |
| | PGRNA- | ACCTCTCTTTACGCGGTCTCC | 17 |
| | X_F, 1528_1548 | | |
| | HX-SHORT- | <5_FAM_ABD>TCTGTGCCT<ZEN>TCTCAT | 18 |
| | 29_FAM_Z-IBFQ | CTGCCGGACCG<IB_FQ> | |
| | HX-SHORT- | <5_HEX_ABD>TCTGTGCCT<ZEN>TCTCAT | 19 |
| | 29_HEX_Z-IBFQ | CTGCCGGACCG<IB_FQ> | |
| | HX-LONG_FAM_Z- | <5_FAM_ABD>CGTCTGTGC<ZEN>CTTCT | 20 |
| | IBFQ | CATCTGCCGGACCG<IB_FQ> | |
| | HX-SHORT-29- | <5_FAM_ABD>CGTCTGTGC<ZEN>CTTCT | 21 |
| | 2_FAM_Z-IBFQ | CATCTGCCGGAC<IB_FQ> | |
| | PGRNA- | <FAM_Thr>CGTCTGT<BHQ_2>GCCTTCTC | 22 |
| | X_FAM_7, 1550_1577 | ATCTGCCGGACCG<Phos> | |
| Other | HC-SHORT-25_FIB | <FAM_Thr>AGAACTCCCTCGCCTCGCAGA | 23 |
| | | CG<IB_FQ> | |
| | HX-SHORT-29_FIB | <FAM_Thr>AGAACTCCCTCGCCTCGCAGA | 24 |
| | | CG<IB_FQ> | |
| | HC-SHORT-25_HIB | <HEX_Thr>AGAACTCCCTCGCCTCGCAGA | 25 |
| | | CG<IB_FQ> | |
| | HX-SHORT-29_HIB | <HEX_Thr>TCTGTGCCTTCTCATCTGCCGG | 26 |
| | | ACCG<IB_FQ> | |

Poly(A)

| | | | SEQ |
|---|---|---|---|
| Target | Oligo Name | Sequence | ID NO: |
| 3′ precore | ESHBVPGRNA_ | ATACGGGTCAATGTCCATGC | 27 |
| or poly(A) | DDPCR_RP3 | | |
| | ESHBVPGRNA_ | GCAACTTTTTCACCTCTGCC | 28 |
| | DDPCR_FP3 | | |
| | ESHBV_HEX_PR3- | <5_HEX_ABD>TTCAAGCCT<ZEN>CCAAG | 29 |
| | Z_IBQ | CTGTGC<IB_FQ> | |
| | ESHBV_FAM_PR3- | <5_FAM_ABD>TTCAAGCCT<ZEN>CCAAG | 30 |
| | Z_IBQ | CTGTGC<IB_FQ> | |
| | HBV_PA | TTTTTTTTTTTTTTTTTTGAAGCTCC | 31 |
| alternates | HBV_PA_MIX | TTTTTTTTTTTTTTTTTTGTAGCTCC | 32 |
| | HBV_PA-Z | TTTTTTTTTTTTTTTTTTGAAGC | 33 |
| | HBV_PA-Z_MIX | TTTTTTTTTTTTTTTTTTGTAGC | 34 |
| | HBV_PA-V | TTTTTTTTTTTTTTTTTTGAAGCTC | 35 |
| | HBV_PA-V_MIX | TTTTTTTTTTTTTTTTTTGTAGCTC | 36 |
| | HBV_PA | TTTTTTTTTTTTTTTTTTGAAGCTCC | 37 |
| | HBV_PA_MIX | TTTTTTTTTTTTTTTTTTGTAGCTCC | 38 |
| | HBV_PA10 | TTTTTTTTTTTTTTGAAGCTCCAA | 39 |
| | HBV_PA10_MIX | TTTTTTTTTTTTTTGTAGCTCCAA | 40 |
| | HBV_PA10_18T | TTTTTTTTTTTTTTTTTTGAAGCTCCAA | 41 |
| | HBV_PA10_18T_MIX | TTTTTTTTTTTTTTTTTTGTAGCTCCAA | 42 |
| other | HBV_PA- | GATCAACGTGTCACCGCCTATTCTAT | 43 |
| | V_5LNA_25_351 | <DLNA_T>TTT<D_LNA_T>TTT<D_LNA_T> | |
| | | TTT<D_LNA_T>T<D_LNA_T>TGAAGCTC | |
| | HBVUP | CATGCAACTTTTTCACCTCTGCCTA | 44 |
| | ESHBVPGRNA_ | <FAM_Thr>TTCAAGCCTCCAAGCTGTGC | 45 |
| | DDPCR_PR3_IBQ | <IB_FQ> | |
| | ESHBVPGRNA_ | <FAM_Thr>TTCAAGCCTCCAAGCTGTGC | 46 |
| | DDPCR_PR3BHQ | <BHQ_2> | |

5′ precore assays

| | | | SEQ |
|---|---|---|---|
| Target | Oligo Name | Sequence | ID NO: |
| 5′precore | HBV-WPC_FP | GGTCTGTTCACCAGCACC | 47 |
| | HBV-WPC_RP | GAAGGAAAGAAGTCAGAAGGCAA | 48 |
| | HBV-RPC_PR-FZIB | <5_FAM_ABD>TTGGGGCAT<ZEN>GGACA | 49 |
| | | TTGACC<IB_FQ> | |
| | HBV-RPC_PRNEW- | <5_HEX_ABD>TTGGGGCAT<ZEN>GGACA | 50 |
| | HZIB | TTGACC<IB_FQ> | |
| Alternates | HBV-JPC_FP | GGTCTGCGCACCAGCACC | 51 |
| | HBV-JPC_RP | GGAAAGAAGTCAGAAGGCAAAAACG | 52 |
| | HBV-RPC_PRLONG- | <5_FAM_ABD>TTTGGGGCA<ZEN>TGGAC | 53 |
| | FZIB | ATTGACCCG<IB_FQ> | |
| | HBV-RPC_PRLONG- | <5_HEX_ABD>TTTGGGGCA<ZEN>TGGAC | 54 |
| | HZIB | ATTGACCCG<IB_FQ> | |
| | HBV-5WANG-PR- | <HEX_Thr>TTGGGGCATGGACATTGACC | 55 |
| | HIB | <IB_FQ> | |
| | HBV-5WANG_PR- | <FAM_Thr>TTGGGGCATGGACATTGACC | 56 |
| | FIB | <IB_FQ> | |

Truncated poly(A)

| | | | SEQ |
|---|---|---|---|
| Target | Oligo name | Sequence | ID NO: |
| truncated | HBV_TR_A_FMIX1 | ACTCTTGGACTCTCAGCAATGT<t_BB_dC> | 57 |
| poly(A) | HBV_TRPA_FL_ | <5_FAM_ABD>ACCGACCTTG<ZEN>AGGC | 58, 138 |
| | FZIB_PR | ATACTTCAAAGACTGTGTGTTTAAAGA | |
| | | <IB_FQ> | |
| | HBV_TRPA_FL_ | <5_HEX_ABD>ACCGACCTTG<ZEN>AGGC | 59, 139 |
| | HZIB_PR | ATACTTCAAAGACTGTGTGTTTAAAGA | |
| | | <IB_FQ> | |
| | HBV_TRPA_7HS | TTTTTTTTTTTTTTTTTTGCTGGTG | 60 |
| putative | BLOCKER_TR1-DD | GTTGCATGGTGCTGGTGAACAGACCAATT | 61 |
| blockers | | TATGCC<Spc_C3> | |
| | BLOCKER_TR3-DD | TAGGCAGAGGTGAAAAAGTTGCATGGTG | 62 |
| | | CTGGTGAACAGACCAATTTATG<Spc_C3> | |
| alternates | HBV_TR_A_FMIX2 | ACTCTTGGACTCTCTGTAATGT<t_BB_dC> | 63 |
| | HBV_TR_A_PMIX1 | <HEX_Thr>ACCGACC<BHQ_2>TTGAGGCA | 64 |
| | | TACTTCAAAGACTGTGTGTTTAAAGA<Phos> | |
| | HBV_TR_A_PMIX2 | <HEX_Thr>ACCTGGA<BHQ_2>TCGAAGA | 65 |
| | | ATACATCAAAGACTGTGTATTTAAGGA<Phos> | |
| | HBV_TRPA_A_10HS_ | TTTTTTTTTTTTTTTTTTGCTGGTGAAC | 66 |
| | MIX1 | | |
| | HBV_TRPA_A_10HS_ | TTTTTTTTTTTTTTTTTTGCTGGTGCGC | 67 |
| | MIX2 | | |
| | HBV_TRPA_14HS | TTTTTTTTTTTTTTTTTTGCTGGTGAACAG | 68 |
| | | AC | |
| | HBV_TRPA_5HS | TTTTTTTTTTTTTTTTTTGCTGG | 69 |
| | HBV_TRPA_33- | <5_FAM_ABD>ACCTTGAGGC<ZEN>ATAC | 70, 140 |
| | 53P_FZIB_PR | TTCAAAGACTGTGTGTTTA<IB_FQ> | |
| | HBV_TRPA_33- | <5_HEX_ABD>ACCTTGAGGC<ZEN>ATAC | 71, 141 |
| | 53P_HZIB_PR | TTCAAAGACTGTGTGTTTA<IB_FQ> | |
| | HBV_TRPA_37- | <5_FAM_ABD>ACCGACCTTG<ZEN>AGGC | 72, 142 |
| | 3P_FZIB_PR | ATACTTCAAAGACTGTGTGTTTA<IB_FQ> | |
| | HBV_TRPA_37- | <5_HEX_ABD>ACCGACCTTG<ZEN>AGGC | 73, 143 |
| | 3P_HZIB_PR | ATACTTCAAAGACTGTGTGTTTA<IB_FQ> | |
| | TR_VB_ANCHORED | GAGACTCGACTCCACAACCA | 74 |
| | TR_VB_TRPA_14HS | GAGACTCGACTCCACAACCATTTTTTTTT | 75 |
| | | TTTTTTTTGCTGGTGAACAGAC | |
| | TR-VB_FORWARD | CCGACCTTGAGGCATACTTC | 76 |
| | TR- | <FAM_Thr>CAGACCAATTTATGCCTACAG | 77 |
| | VB_PROBE_V_FULL | CCTCC<BHQ_1> | |
| | HBV_TR_X_FORWA | <pdU>TGAGGCA<pdU>A<5_Me_dC>T<pdU> | 78 |
| | RD | <5_Me_dC>AAAGA<5_Me_dC>TG | |
| | HBV_TR_X_PROBE | <HEX_Thr>TG<pdU>AC<pdU>AGGA<BHQ2> | 79, 144 |
| | | GG<5_Me_dC>TGTAGG<5_Me_dC>ATAA | |
| | | ATTG<Phos> | |

Splice variants

| | | | SEQ |
|---|---|---|---|
| Target | Oligo Name | Sequence | ID NO: |
| SP1 | HBVRSP_1_117_FP | CGTCGCAGAAGATCTCAAT | 80 |
| | HBVRSP_1_117_PR_ | <5_FAM_ABD>CTCGGGAAT<ZEN>CTCAA | 81 |
| | FZIB | TGAACATCA<IB_FQ> | |
| | HBVRSP_1_117_PR_ | <5_FAM_ABD>TGTCCTGGC<ZEN>CAAAA | 82 |
| | HZIB | TTCGCA<IB_FQ> | |
| | HBVRSP_1_117_RP | GTTCCTTGAGCAGGAATCG | 83 |
| SP2 | HBVRSP_2_50_FP | TTAGAGTCTCCGGAACATTG | 84 |
| | HBVRSP_2_50_PR_ | <5_FAM_ABD>ATACAGCAC<ZEN>TCAGA | 85 |
| | FZIB | CGACGA<IB_FQ> | |
| | HBVRSP_2_50_PR_ | <5_HEX_ABD>ATACAGCAC<ZEN>TCAGA | 86 |
| | HZIB | CGACGA<IB_FQ> | |
| | HBVRSP_2_50_RP | CGAGGGAGTTCTTCTTCTAGG | 87 |
| SP3 | HBVRSP_3_718- | TTCCTTGAGCAGGAATCGT | 88 |
| | 2_RP | | |
| | HBVRSP_3_718_FP | CATTGTTCACCTCACCATACA | 89 |
| | HBVRSP_3_718_PR_ | <5_FAM_ABD>CTCAGGAAC<ZEN>ATCAA | 90 |
| | FZIB | CTACCAGC<IB_FQ> | |
| | HBVRSP_3_718_PR_ | <5_HEX_ABD>CTCAGGAAC<ZEN>ATCAA | 91 |
| | HZIB | CTACCAGC<IB_FQ> | |
| | HBVRSP_3_718_RP | GTTCCTTGAGCAGGAATCGT | 92 |
| SP4 | HBVRSP_4_349_FP | CATTGTTCACCTCACCATAC | 93 |
| | HBVRSP_4_349_PR_ | <5_FAM_ABD>TTGGGACGA<ZEN>CGAGG | 94 |
| | FZIB | CAGG<IB_FQ> | |
| | HBVRSP_4_349_PR_ | <5_HEX_ABD>TTGGGACGA<ZEN>CGAGG | 95 |
| | HZIB | CAGG<IB_FQ> | |
| | HBVRSP_4_349_RP | CGAGGGAGTTCTTCTTCTAGG | 96 |
| SP5 | HBVRSP_5_582- | GTTCCTTGAGCAGGAATC | 97 |
| | 2_RP | | |
| | HBVRSP_5_582- | TTCCTTGAGCAGGAATCG | 98 |
| | 3_RP | | |
| | HBVRSP_5_582-FP | AGCACTCAGGCAAGCTAT | 99 |
| | HBVRSP_5_582_ | <5_FAM_ABD>TGGGAACAT<ZEN>CAACT | 100 |
| | FZIB | ACCAGC<IB_FQ> | |
| | HBVRSP_5_582_ | <5_HEX_ABD>TGGGAACAT<ZEN>CAACT | 101 |
| | HZIB | ACCAGC<IB_FQ> | |
| | HBVRSP_5_582_RP | GTTCCTTGAGCAGGAATCGT | 102 |
| Paper | HBV_PSP-2_FP | CGCGTCGCAGAAGATCT | 103 |
| | HBV_PSP-2_RP | ATGGGAATACAGGTGCAATTTC | 104 |
| | HBV_PSP_FP | CCGCGTCGCAGAAGATCT | 105 |
| | HBV_PSP_PR_FZIB | <5_FAM_ABD>ATCTCGGGA<ZEN>TCTCA | 106 |
| | | ATGAACATC<IB_FQ> | |
| | HBV_PSP_PR_HZIB | <5_HEX_ABD>ATCTCGGGA<ZEN>TCTCA | 107 |
| | | ATGAACATC<IB_FQ> | |
| | HBV_PSP_RP | ATGGGAATACAGGTGCAATTTCC | 108 |
| Paper WT | HBV_PWT-2_FP | TAGACTCGTGGTGCAGTTCT | 109 |
| | HBV_PWT-2_RP | ATAGCAGCAGGATGAAGCG | 110 |
| | HBV_PWT_FP | TCTAGACTCGTGGTGCAGTTCTCTC | 111 |
| | HBV_PWT_PR2_ | <5_FAM_ABD>TCGCAGTCC<ZEN>CCAAC | 112 |
| | FZIB | CTCCAATC<IB_FQ> | |
| | HBV_PWT_PR2_ | <5_HEX_ABD>TCGCAGTCC<ZEN>CCAAC | 113 |
| | HZIB | CTCCAATC<IB_FQ> | |
| | HBV_PWT_PR_FZIB | <5_FAM_ABD>TGTCCTGGC<ZEN>CAAAA | 114 |
| | | TTCGCA<IB_FQ> | |
| | HBV_PWT_PR_ | <5_HEX_ABD>TGTCCTGGC<ZEN>CAAAA | 115 |
| | HZIB | TTCGCA<IB_FQ> | |
| | HBV_PWT_RP | CATAGCAGCAGGATGAAGCGGAA | 116 |

S gene

| | | | SEQ |
|---|---|---|---|
| Target | Oligo Name | Sequence | ID NO: |
| s1000 | HBV_S1000_FP-1-1 | GCACCTGTATTCCCATCCCATC | 117 |
| assay | HBV_S1000_PR-1- | <5_FAM_ABD>AGAAACGGA<ZEN>CTGA | 118 |
| | 1_FZI | GGCCCACTCCCATAGG<IB_FQ> | |
| | HBV_S1000_PR-1- | <5_HEX_ABD>AGAAACGGA<ZEN>CTGAG | 119 |
| | 1_HZI | GCCCACTCCCATAGG<IB_FQ> | |
| | HBV_S1000_RP-1-1 | CAAATGGCACTAGTAAACTGAGCCA | 120 |
| s600 assay | HBV_S600_FP-1- | CG<7_Dz_dG>GGTTTTTCTTGTTGACAAGA | 121 |
| | 1_7G2 | ATC | |
| | HBV_S600_FP-1- | CGG<7_Dz_dG>GTTTTTCTTGTTGACAAGA | 122 |
| | 1_7G3 | ATC | |
| | HBV_S600_PR-1- | <5_FAM_ABD>AGTCCACCA<ZEN>CGAGT | 123 |
| | 1_FZI | CTAGACTCTGTGGTATTGTGAG<IB_FQ> | |
| | HBV_S600_PR-1- | <5_HEX_ABD>AGTCCACCA<ZEN>CGAGT | 124 |
| | 1_HZI | CTAGACTCTGTGGTATTGTGAG<IB_FQ> | |
| | HBV_S600_RP-1- | GAGGTTG<7_Dz_dG>GGACTGCGAA | 125 |
| | 1_7G2 | | |
| | HBV_S600_RP-1- | GAGGTTGG<7_Dz_dG>GACTGCGAA | 126 |
| | 1_7G3 | | |
| s1000 | HBV_S1000_PR-1- | <5_FAM_ABD>AGAAACGG<BHQ_2>ACTG | 127 |
| assay | 1_FAM | AGGCCCACTCCCATAGG<Spc_C3> | |
| | HBV_S1000_PR-1- | <5_HEX_ABD>AGAAACGG<BHQ_2>ACTG | 128 |
| | 1_HEX | AGGCCCACTCCCATAGG<Spc_C3> | |
| | HBV_S1000_RP-2-1 | CTGAACAAATGGCACTAGTAAACTGA | 129 |
| | HBV_S1000_RP-3-1 | ACCACTGAACAAATGGCACTAGTAAA | 130 |
| S600 assay | HBV_S600_FP-1- | CG<7_Dz_dG>GGTTTTTCTTGTTGACAAGA | 131 |
| | 1_7G2 | ATC | |
| | HBV_S600_FP-2- | G<7_Dz_dG>GGTTTTTCTTGTTGACAAGAA | 132 |
| | 1_7G2 | TCC | |
| | HBV_S600_FP-2- | GG<7_Dz_dG>TTTTTCTTGTTGACAAGAAT | 133 |
| | 1_7G3 | CC | |
| | HBV_S600_PR-1-1 | <5_FAM_ABD>AGTCCACC<BHQ_2>ACGA | 134 |
| | | GTCTAGACTCTGTGGTATTGTGAG<Spc_C3> | |
| | HBV_S600_PR-1- | <5_HEX_ABD>AGTCCACC<BHQ_2>ACGA | 135 |
| | 1_HEX | GTCTAGACTCTGTGGTATTGTGAG<Spc_C3> | |
| | HBV_S600_RP-2- | TGGAGGTTG<7_Dz_dG>GGACTGC | 136 |
| | 1_7G2 | | |
| | HBV_S600_RP-2- | TGGAGGTTGG<7_Dz_dG>GACTGC | 137 |
| | 1_7G3 | | |

### Example 2

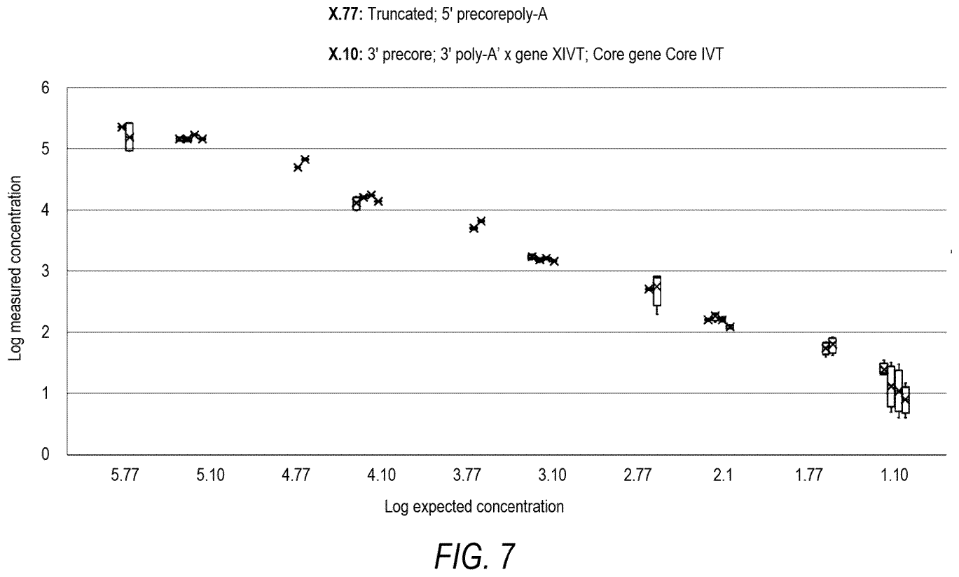

The present example illustrates an HBV dPCR assay according to the present disclosure. An exemplary digital PCR assay for detecting HBV RNAs can be designed to detect the presence or absence of one or more HBV targets as shown in FIG. 1, including precore mRNA 5′ end (non-pgRNA) 1, core target 2, X gene target 3, truncated RNA 3′ end (poly(A) junction) 4, precore/core target 5, full-length RNA 3′ end (poly(A) junction) 6, selected splice junctions (examples shown) 7, S gene, pre-splice site 8, S gene, post-splice site 9, and pgRNA+pc-mRNA 5′ end for 3.5 kb transcripts 10. In the present example primers and probes were designed against six HBV targets in the form of i) 3′ precore, ii) 3′ poly(A), iii) X gene mRNA, iv) core gene mRNA, v) truncated poly(A) and vi) 5′-precore. A dilution series of known concentrations of each of the six targets was prepared and a ddPCR assay comprising primers and probes from Table 4 was implemented to detect each of the six targets. Measured concentrations for each of the six targets in the samples were calculated and plotted against the known (expected) concentrations for those samples as shown in linearity plot in FIG. 6 and box and whisker plot of FIG. 7. Expected concentrations correlated strongly with measured concentrations as evidenced by linear regression data as shown in Table 5.

TABLE 5

| HBV Target | slope | intercept | R² |
|---|---|---|---|
| 3′ precore | 0.9451 | 0.2902 | 0.9961 |
| 3′ poly(A) | 1.0008 | 0.0840 | 0.9898 |
| X gene (X IVT) | 1.0388 | 0.0312 | 0.9900 |
| Core gene (Core IVT) | 1.0563 | 0.1825 | 0.9947 |
| Truncated poly(A) | 0.9556 | 0.0687 | 0.9954 |
| 5′ precore | 0.9222 | 0.2341 | 0.9664 |

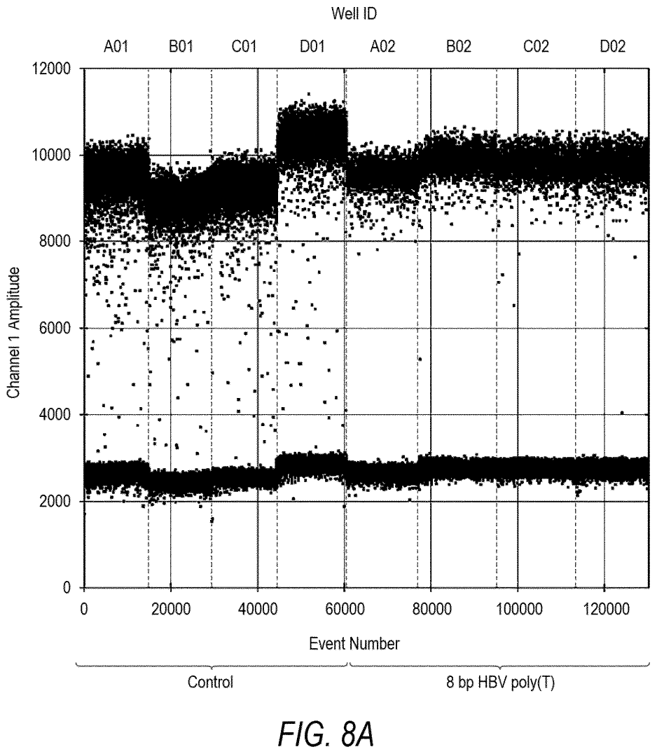

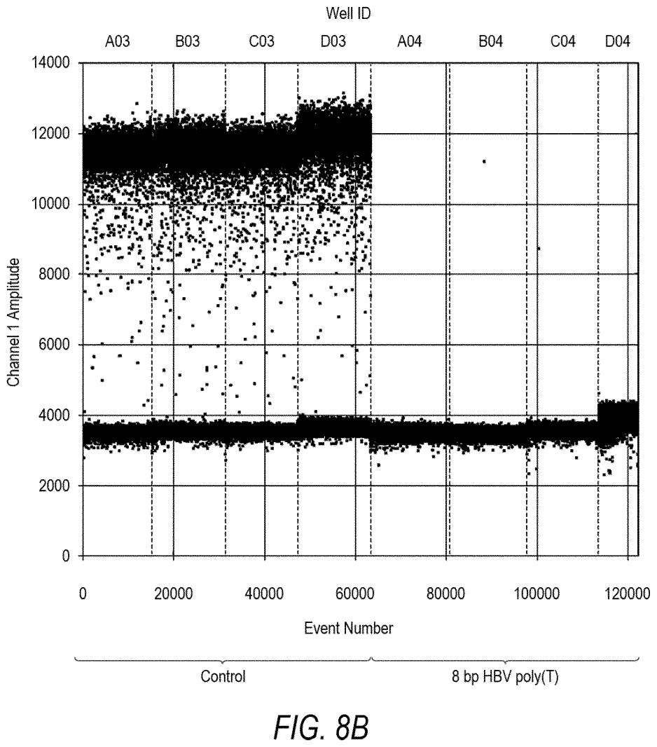

Turning to FIGS. 8A and 8B, a comparison of dPCR assay results is shown for amplification of 3′ precore RNA (FIG. 8A) and 3′ precore DNA plasmid (FIG. 8B) using either a control reverse primer complementary to precore HBV (excluding the poly(A) tail) or an 8 bp poly(T) containing reverse primer complementary to precore HBV (including the poly(A) tail). As shown in FIG. 8A, experiments with both the control and poly(T) primers in sample comprising 3′ precore HBV RNA were successful for generating a detectable fluorescence signal around 10,0000 units above the baseline signal amplitude of around 2,500 units. As expected, experiments with samples comprising 3′ precore HBV DNA resulted in a positive signal around 12,000 units relative to a baseline of around 3,500 units only for the control primer (FIG. 8B) as the 3′ precore HBV DNA template does not include a poly(A) tail complementary to the poly(T) amplification primer.

### Example 3

The present example illustrates sequences of oligonucleotides used in HBV dPCR assays directed to the S gene. Two S gene assays were designed to accommodate the impact of possible splicing: S-gene Assay(S) and S-gene Assay, post-splice (SPS). For the S gene assay, specific primers (HBV_S600_FP-1-1_7G2 (SEQ ID NO:121) and HBV_S600_RP-1-1_7G2 (SEQ ID NO:125)) and a probe (HBV_S600_PR-1-1_FZI (SEQ ID NO:123) were employed. The SPS assays implemented three different combinations of primers and probes: i) SPS Assay-1 (HBV_S1000_FP-1-1 (SEQ ID NO: 117) and HBV_S1000_RP-1-1 (SEQ ID NO:120)), ii) SPS Assay-2 (HBV_S1000_FP-1-1 (SEQ ID NO: 117) and HBV_S1000_RP-2-1 (SEQ ID NO:129)), and iii) SPS Assay-3 (HBV_S1000_FP-1-1 (SEQ ID NO:117) and HBV_S1000_RP-3-1 (SEQ ID NO:130)). Of note, a common probe, HBV_S1000_PR-1-1_FZI (SEQ ID NO:118), was used for all three SPS assays.

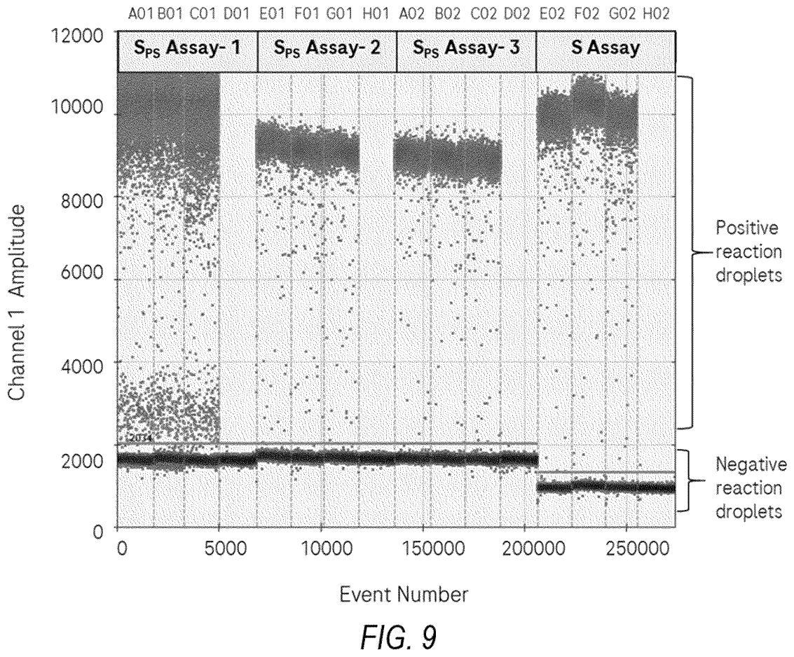

The template for these assays was the HBV pgRNA in vitro transcript (pgRNA IVT). The first experiment focused on conducting preliminary testing with S-gene assays with the specific purpose of implementing two assay designs that could accommodate the potential impact of splicing. With reference to FIG. 9, both the S-gene(S) assay and the S-gene Post-Splice (SPS) assay successfully detected target regions as intended. While SPS assay 1 exhibited a second positive droplet band at a lower channel 1 amplitude, SPS assays 1 and 3, as well as the S assay, demonstrated optimal performance, exhibiting only a single positive droplet band with minimal background signal. In conclusion, the present S-gene assays were effective for specifically detecting two adjacent regions within the S-gene sequence.

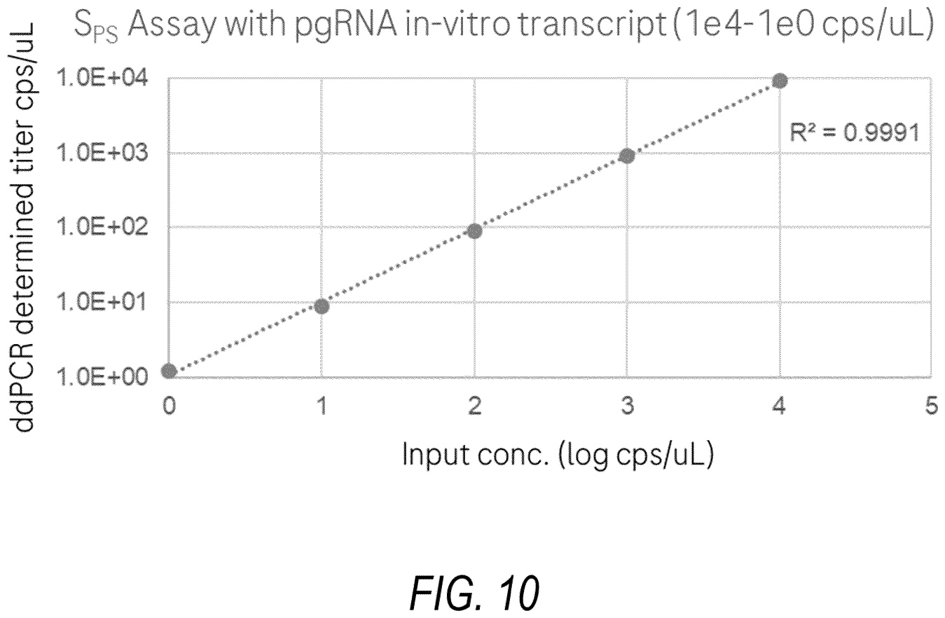

Results from FIG. 10 illustrate the outcome of sensitivity testing for SPS assay 2. Previous testing patterns persisted, with reactions involving pgRNA IVT at concentrations of 10⁶ and 10⁵ copies per microliter (cps/μL) reaching saturation. Of note, one of the replicates for IVT at 10³ cps/μL displayed lower fluorescence, and was therefore omitted from titer calculations. The assay demonstrated sensitivity in detecting the template even at a concentration as low as 1 cps/μL.

### Example 4

The present example illustrates HBV assays directed to the pre-splice site of the core region while avoiding major downstream spliced introns.

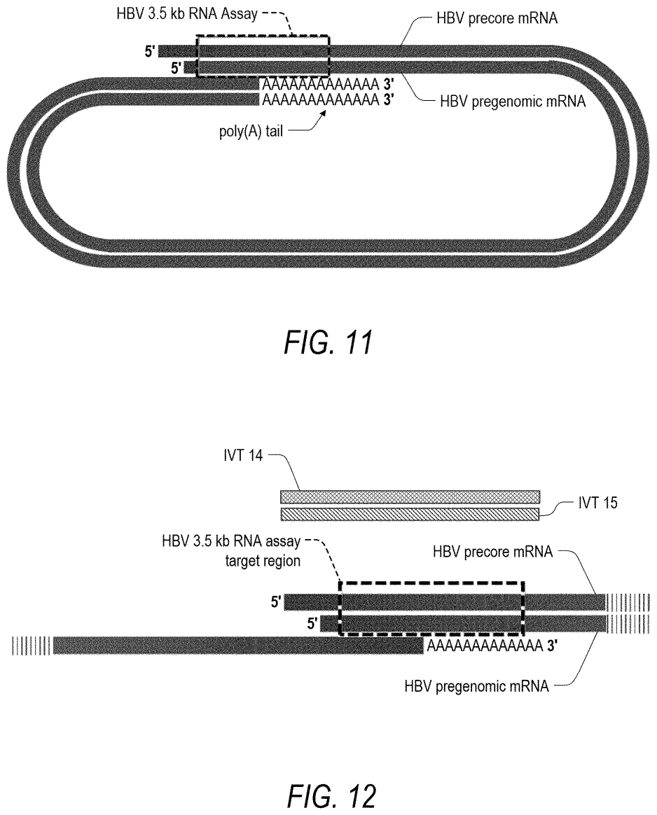

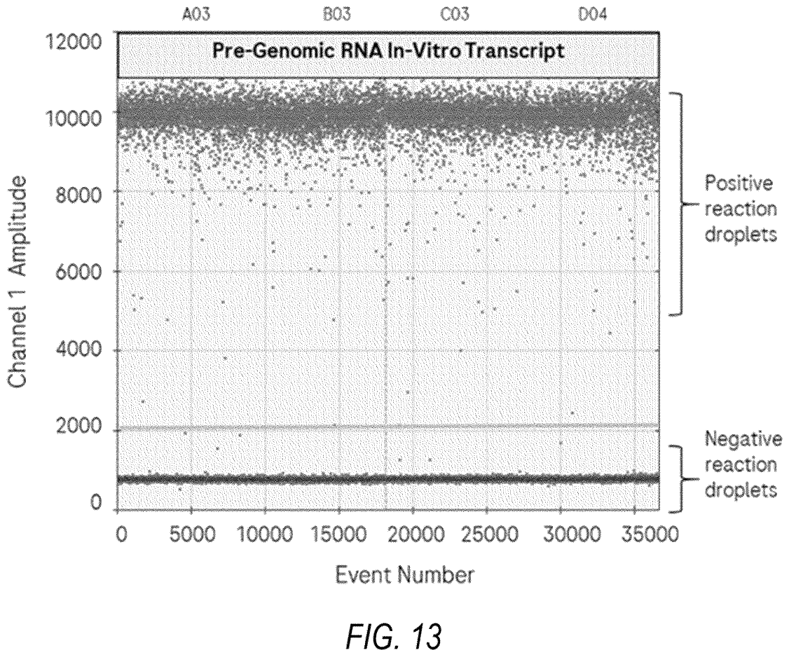

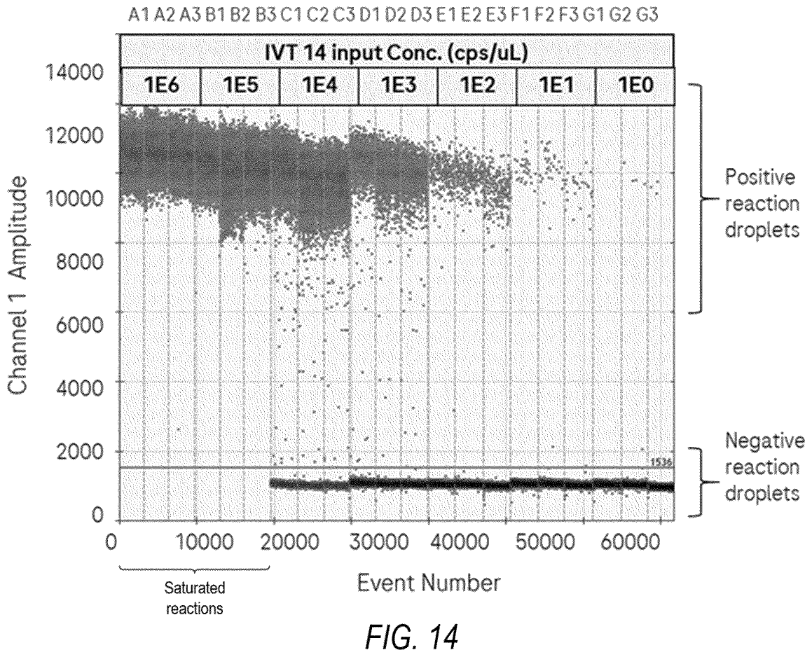

The HBV 3.5 kb RNA assay targets the pre-splice site of core region and strategically avoiding major downstream spliced introns. This design allows for the detection of both pgRNA and the slightly longer precore mRNA as illustrated in FIG. 11. With reference to FIG. 12, the assay was designed and tested with two similar templates (IVT 14 and IVT 15), each of which comprise at least a portion of the 5′ precore region representing precore RNA, as well as pgRNA IVT. Successful detection of IVT 14, IVT 15, and pgRNA was achieved by ddPCR (FIGS. 13 and 14).

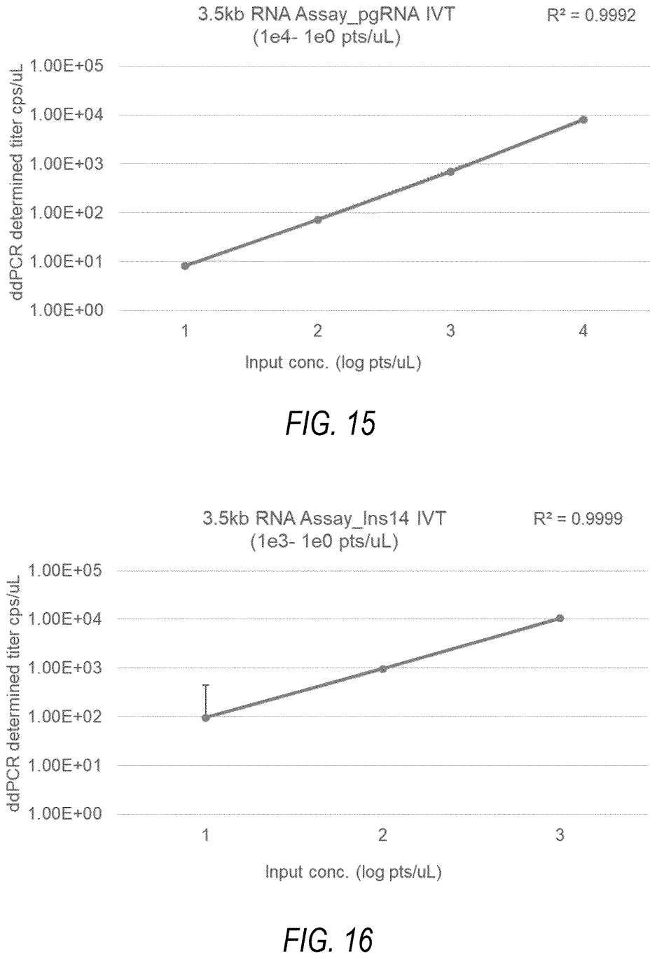

Turning to FIG. 14, further sensitivity testing revealed that the 3.5 kb RNA assay of the present example demonstrated no significant background noise or rain with the IVT 14 template. At assay input concentrations of 10⁵ copies/μL and higher, ddPCR reactions with the IVT 14 template were observed to be saturated. As expected, no significant positive droplets were observed with templates representative of the 3′ end of the HBV mRNA due to the absence of a binding region for the reverse primer. Similar results were observed for the IVT 15 template (not shown). Linearity testing, depicted in FIGS. 15 and 16, indicated that the present assay was sensitive enough to detect from 10⁴ copies/μL down to 10¹ copies/μL of pgRNA and from 10³ copies/μL down to 10¹ copies/μL of IVT 14, with relatively low standard deviation within these concentrations. Reactions tested at higher template concentration (i.e., >10⁴ copies/μL of pgRNA and >10³ copies/μL of IVT 14) resulted in saturation, with no negative reaction droplets observed.

### Example 5

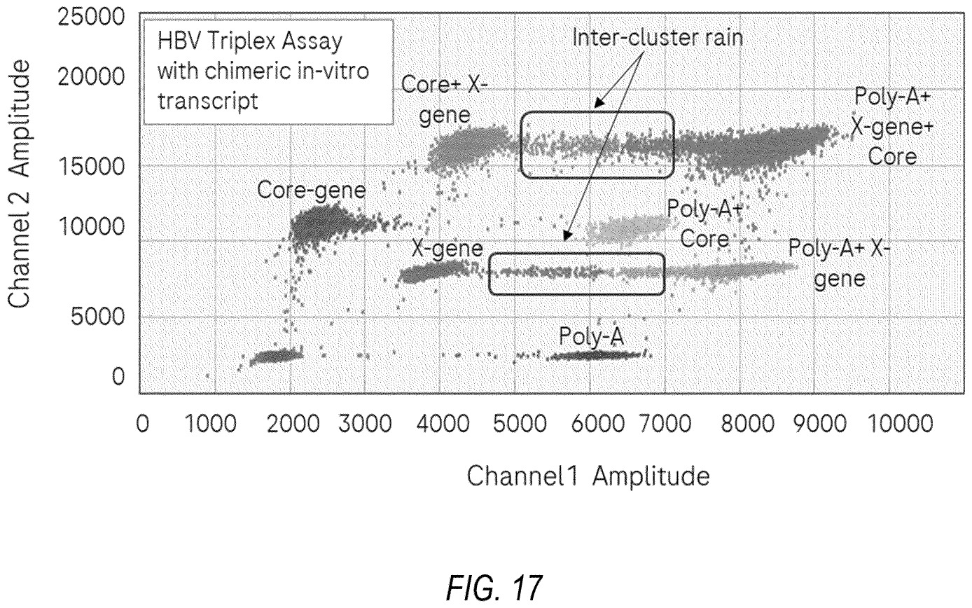

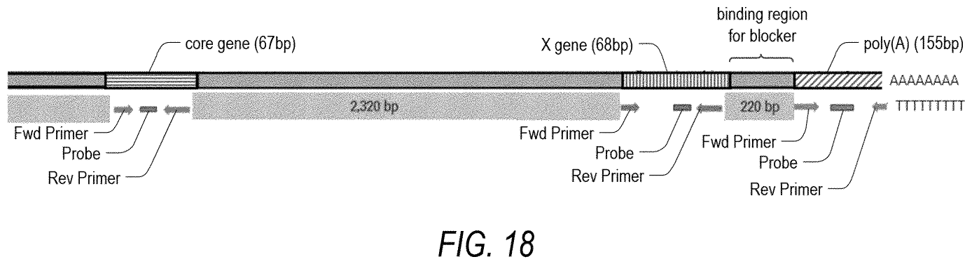

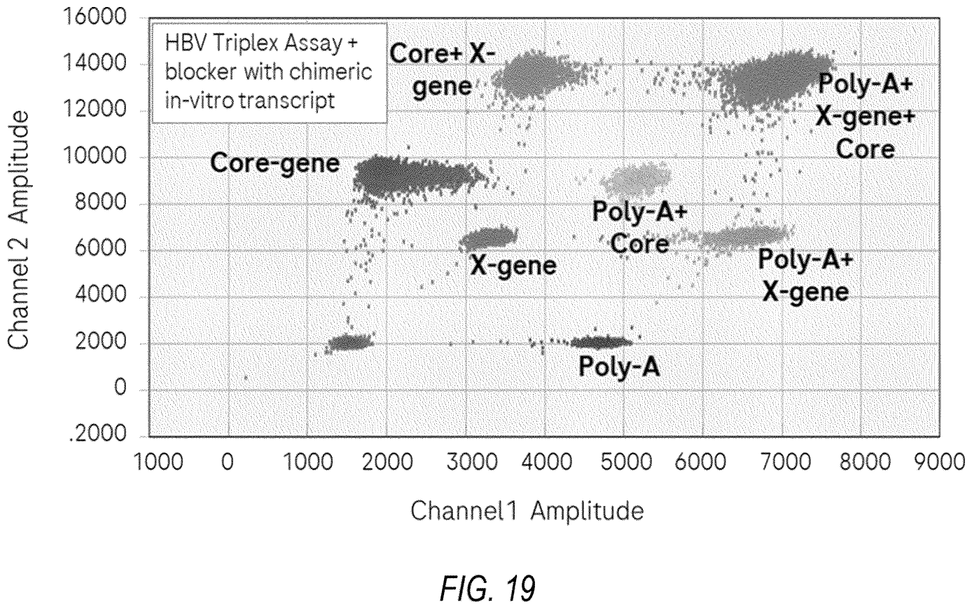

The present example illustrates HBV triplex assays including a blocker according to the present disclosure. Initial ddPCR multiplex assays directed towards detection of at least core, X gene, and poly(A) targets resulted in the observation of inter-cluster rain (FIG. 17). On closer inspection, this inter-cluster rain was determined to be localized to the clusters containing X gene and poly(A) targets. To reduce or eliminate the occurrence of the observed inter-cluster rain, oligonucleotides incapable of extension of DNA polymerase were designed for binding to the region between X gene and poly(A), with the goals of i) preventing the formation of dual-target amplicons (e.g., an amplicon including both X gene and poly(A) regions), and ii) fostering the production of single-target amplicons, as illustrated in FIG. 18. The non-extendable blockers oligonucleotides were strategically designed to bind to the region between X gene and poly(A), blocking the potential formation of hybrid amplicons involving X gene and poly(A). The initial ddPCR multiplex assays targeting core, X gene and poly(A) were repeated with the addition of blocker oligonucleotides present at the same concentration as the primers. The ddPCR assay data, depicted in FIG. 19, illustrated that the presence of the blocker oligonucleotides resulted a significant reduction in inter-cluster rain.

### Example 6

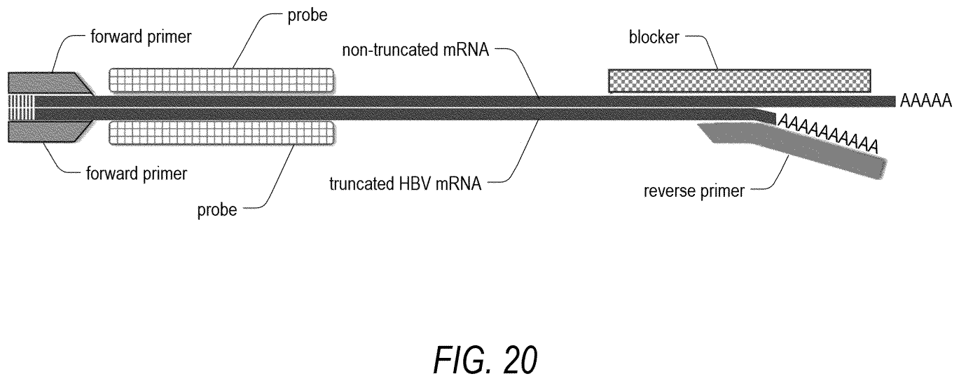

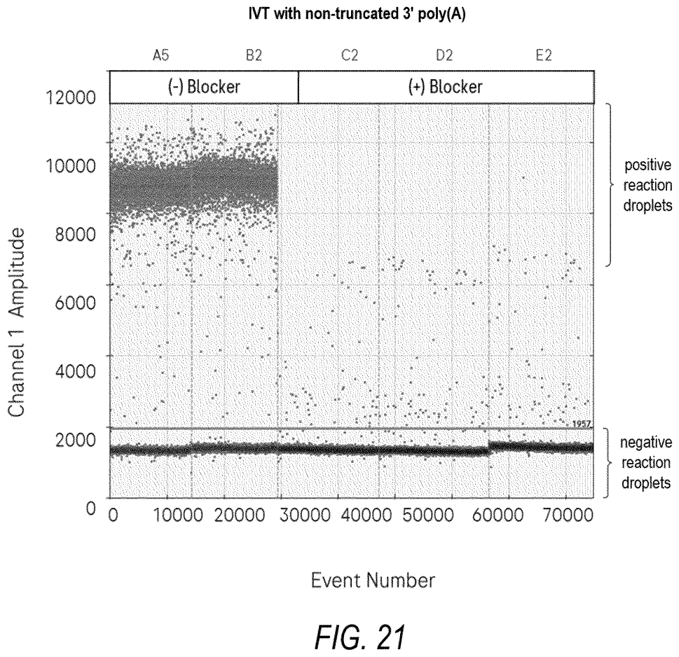

The present example illustrates an HBV assay for detection of truncated poly(A) according to the present disclosure. With reference to FIG. 20, a truncated poly(A) assay was developed with the goal of detecting truncated HBV RNA species characterized by earlier poly(A) regions. The assay included forward primer HBV_TR_A_FMIX1-N (SEQ ID NO:57), reverse primer HBV_TRPA_7HS (SEQ ID NO: 60), probe HBV_TRPA_FL_FZIB_PR (SEQ ID NO:58) and blocker oligonucleotide TR3_DD (SEQ ID NO:62). Similar to the application described in FIGS. 4 and 18, blocker oligonucleotide TR3_DD served the role of impeding the binding of the truncated poly(A) reverse primer to its corresponding target region in the non-truncated HBV RNA and/or HBV DNA. The results the ddPCR assay comprising the aforementioned primers, probe and blocker indicated that amplification of the in vitro transcript with non-truncated 3′ poly(A) (FIG. 21) was effectively inhibited, whereas amplification was successfully achieved for the in vitro transcript comprising the truncated 3′ poly(A) region.

### Example 7

The present example illustrates further sequences of oligonucleotides used in HBV dPCR assays. The nucleotide sequences and descriptions of the primer (forward and reverse), probe and blocking oligonucleotides used to perform the dPCR assays for the detection and quantitation of various HBV RNA forms and gene targets are listed in Table 6.

TABLE 6

| | | | SEQ ID |
|---|---|---|---|
| Assay | Target | Sequence | NO(s): |
| 3′ precore | Precore poly(A), | T<D_LNA_T>TTT<D_LNA_T>TTT<D_LNA_T> | 145 |
| | 7 bp HBV | TTT<D_LNA_T>T<D_LNA_T>TGAAGCTC | |
| 3′ precore | Precore poly(A), | T<D_LNA_T>T<D_LNA_T>T<D_LNA_T>T | 146 |
| | 7 bp HBV | <D_LNA_T>T<D_LNA_T>T<D_LNA_T>T | |
| | | <D_LNA_T>T<D_LNA_T>TGAAGCTC | |
| 3′ precore | Precore poly(A), | GATCAACGTGTCACCGCCTT<D_LNA_T>TTT | 147 |
| | 7 bp HBV | <D_LNA_T>TTT<D_LNA_T>TTT<D_LNA_T>T | |
| | | <D_LNA_T>TGAAGCTC | |
| 3′ precore | Precore poly(A), | GATCAACGTGTCACCGCCTT<D_LNA_T>T<D_ | 148 |
| | 7 bp HBV | LNA_T>T<D_LNA_T>T<D_LNA_T>T<D_LN_ | |
| | | A_T>T<D_LNA_T>T<D_LNA_T>T<D_LNA_T> | |
| | | TGAAGCTC | |
| 3′ precore | Precore poly(A), | GATCAACGTGTCACCGCCTATTCTAT<D_LN_ | 149 |
| | 7 bp HBV | A_T>TTT<D_LNA_T>TTT<D_LNA_T>TTT<D_ | |
| | | LNA_T>T<D_LNA_T>TGAAGCTC | |
| 3′ precore | Precore poly(A), | T<D_LNA_T>TTT<D_LNA_T>TTT<D_LNA_T> | 150 |
| | 7 bp HBV | TTT<D_LNA_T>T<D_LNA_T>TGAA<D_LNA_ | |
| | | G>CTC | |
| 3′ precore | Precore poly(A), | T<D_LNA_T>TTT<D_LNA_T>TTT<D_LNA_T> | 151 |
| | 8 bp HBV | TTT<D_LNA_T>T<D_LNA_T>TGAAGCTCC | |
| 3′ precore | Precore poly(A), | T<D_LNA_T>T<D_LNA_T>T<D_LNA_T>T<D_ | 152 |
| | 8 bp HBV | LNA_T>T<D_LNA_T>T<D_LNA_T>T<D_LNA_ | |
| | | T>T<D_LNA_T>TGAAGCTCC | |
| 3′ precore | Precore poly(A), | GATCAACGTGTCACCGCCTT<D_LNA_T>TTT | 153 |
| | 8 bp HBV | <D_LNA_T>TTT<D_LNA_T>TTT<D_LNA_T>T | |
| | | <D_LNA_T>TGAAGCTCC | |
| 3′ precore | Precore poly(A), | GATCAACGTGTCACCGCCTT<D_LNA_T>T<D_ | 154 |
| | 8 bp HBV | LNA_T>T<D_LNA_T>T<D_LNA_T>T<D_LN_ | |
| | | A_T>T<D_LNA_T>T<D_LNA_T>T<D_LNA_T> | |
| | | TGAAGCTCC | |
| 3′ precore | Precore poly(A), | GATCAACGTGTCACCGCCTATTCTA<D_LNA_ | 155 |
| | 8 bp HBV | T>TTT<D_LNA_T>TTT<D_LNA_T>TTT<D_ | |
| | | LNA_T>T<D_LNA_T>TGAAGCTCC | |
| 3′ precore | Precore poly(A), | T<D_LNA_T>TTT<D_LNA_T>TTT<D_LNA_T> | 156 |
| | 8 bp HBV | TTT<D_LNA_T>T<D_LNA_T>TGAA<D_LNA_ | |
| | | G>CTCC | |
| 3′ precore | Precore poly(A), | T<D_LNA_T>TTT<D_LNA_T>TTT<D_LNA_T> | 157 |
| | 9 bp HBV | TTT<D_LNA_T>T<D_LNA_T>TGAAGCTCCA | |
| 3′ precore | Precore poly(A), | T<D_LNA_T>T<D_LNA_T>T<D_LNA_T>T<D_ | 158 |
| | 9 bp HBV | LNA_T>T<D_LNA_T>T<D_LNA_T>T<D_LNA_ | |
| | | T>T<D_LNA_T>TGAAGCTCCA | |
| 3′ precore | Precore poly(A), | GATCAACGTGTCACCGCCTT<D_LNA_T>TTT | 159 |
| | 9 bp HBV | <D_LNA_T>TTT<D_LNA_T>TTT<D_LNA_T>T | |
| | | <D_LNA_T>TGAAGCTCCA | |
| 3′ precore | Precore poly(A), | GATCAACGTGTCACCGCCTT<D_LNA_T>T<D_ | 160 |
| | 9 bp HBV | LNA_T>T<D_LNA_T>T<D_LNA_T>T<D_LN_ | |
| | | A_T>T<D_LNA_T>T<D_LNA_T>T<D_LNA_T> | |
| | | TGAAGCTCCA | |
| 3′ precore | Precore poly(A), | GATCAACGTGTCACCGCCTATTCTA<D_LNA_ | 161 |
| | 9 bp HBV | T>TT<D_LNA_T>TTT<D_LNA_T>TTT<D_LN_ | |
| | | A_T>T<D_LNA_T>TGAAGCTCCA | |
| 3′ precore | Precore poly(A), | T<D_LNA_T>TTT<D_LNA_T>TTT<D_LNA_T> | 162 |
| | 9 bp HBV | TTT<D_LNA_T>T<D_LNA_T>TGAA<D_LNA_ | |
| | | G>CTCCA | |
| 3′ precore | Precore poly(A), | T<D_LNA_T>TTT<D_LNA_T>TTT<D_LNA_T> | 163 |
| | 10 bp HBV | TTT<D_LNA_T>T<D_LNA_T>TGAAGCTCCA | |
| | | A | |
| 3′ precore | Precore poly(A), | T<D_LNA_T>T<D_LNA_T>T<D_LNA_T>T<D_ | 164 |
| | 10 bp HBV | LNA_T>T<D_LNA_T>T<D_LNA_T>T<D_LNA_ | |
| | | T>T<D_LNA_T>TGAAGCTCCAA | |
| 3′ precore | Precore poly(A), | GATCAACGTGTCACCGCCTT<D_LNA_T>TTT | 165 |
| | 10 bp HBV | <D_LNA_T>TTT<D_LNA_T>TTT<D_LNA_T>T | |
| | | <D_LNA_T>TGAAGCTCCAA | |
| 3′ precore | Precore poly(A), | GATCAACGTGTCACCGCCTATTCTA<D_LNA_ | 166 |
| | 10 bp HBV | T>TT<D_LNA_T>TT<D_LNA_T>TTT<D_LNA_ | |
| | | T>T<D_LNA_T>TGAAGCTCCAA | |
| 3′ precore | Precore poly(A), | GATCAACGTGTCACCGCCTT<D_LNA_T>T<D_ | 167 |
| | 10 bp HBV | LNA_T>T<D_LNA_T>T<D_LNA_T>T<D_LN_ | |
| | | A_T>T<D_LNA_T>T<D_LNA_T>T<D_LNA_T> | |
| | | TGAAGCTCCAA | |
| 3′ precore | Precore poly(A), | T<D_LNA_T>TTT<D_LNA_T>TTT<D_LNA_T> | 168 |
| | 10 bp HBV | TTT<D_LNA_T>T<D_LNA_T>TGAA<D_LNA_ | |
| | | G>CTCCAA | |
| Core and X | Core gene | <CY5>AGAACTCCCT<BHQ_2>CGCCTCGCAG | 169, 170 |
| gene | | ACG<HEG><Spc_C3> | |
| Core and X | Core gene | <CY5.5>AGAACTCCCT<BHQ_2>CGCCTCGCA | 169, 170 |
| gene | | GACG<Spc_C3> | |
| Core and X | Core gene | <5TEX_615>AGAACTCCCT<BHQ_2>CGCCTC | 169, 170 |
| gene | | GCAGACG<Spc_C3> | |
| 3′ precore | Precore poly(A) | <CY5.5>TTCAAGCCTC<BHQ_2>CAAGCTGTG | 171, 172 |
| | | C<Spc_C3> | |
| 3′ precore | Precore poly(A) | <CY5>TTCAAGCCTC<BHQ_2>CAAGCTGTGC | 171, 172 |
| | | <Spc_C3> | |
| 3′ precore | Precore poly(A) | <5_FAM_ABD>TTCAAGCC<BHQ_2>TCCAAG | 173 |
| | | CTGTGC<Spc_C3> | |
| 3′ precore | Precore poly(A) | <5_HEX_ABD>TTCAAGCC<BHQ_2>TCCAAG | 173 |
| | | CTGTGC<Spc_C3> | |
| 3′ precore | Precore poly(A) | <5TEX_615>TTCAAGCCTC<BHQ_2>CAAGCT | 171, 172 |
| | | GTGC<Spc_C3> | |
| Core and X | Core gene | <5_FAM_ABD>AGAACTC<BHQ_2>CCTCGCC | 174 |
| gene | | TCGCAGACGAAG<Spc_C3> | |
| Core and X | Core gene | <5_FAM_ABD>AGAACTCC<BHQ_2>CTCGCC | 175 |
| gene | | TCGCAGACG<Spc_C3> | |
| Core and X | Core gene | <5_HEX_ABD>AGAACTCC<BHQ_2>CTCGCC | 175 |
| gene | | TCGCAGACG<Spc_C3> | |
| Core and X | Core gene | <CY5.5>AGAACTCCCT<BHQ_2>CGCCTCGCA | 169, 176 |
| gene | | GACGAAG<Spc_C3> | |
| Core and X | Core gene | <CY5>AGAACTCCCT<BHQ_2>CGCCTCGCAG | 169, 176 |
| gene | | ACGAAG<Spc_C3> | |
| Core and X | Core gene | <5TEX_615>AGAACTCCCT<BHQ_2>CGCCTC | 169, 176 |
| gene | | GCAGACGAAG<Spc_C3> | |
| Core and X | X gene | <5_HEX_ABD>CGTCTGT<BHQ_2>GCCTTCTC | 177 |
| gene | | ATCTGCCGGACCG<Spc_C3> | |
| Core and X | X gene | <CY5>TCTGTGCCTT<BHQ_2>CTCATCTGCC | 178, 179 |
| gene | | GGACCG<Spc_C3> | |
| Core and X | X gene | <CY5.5>TCTGTGCCTT<BHQ_2>CTCATCTGC | 178, 179 |
| gene | | CGGACCG<Spc_C3> | |
| Core and X | X gene | <5_FAM_ABD>TCTGTGCC<BHQ_2>TTCTCAT | 180 |
| gene | | CTGCCGGACCG<Spc_C3> | |
| Core and X | X gene | <5_HEX_ABD>TCTGTGCC<BHQ_2>TTCTCAT | 180 |
| gene | | CTGCCGGACCG<Spc_C3> | |
| Core and X | X gene | <5TEX_615>TCTGTGCCTT<BHQ_2>CTCATCT | 178, 179 |
| gene | | GCCGGACCG<Spc_C3> | |
| Core and X | X gene | <CY5.5>CGTCTGTGCC<BHQ_2>TTCTCATCT | 180, 181 |
| gene | | GCCGGACCG<Spc_C3> | |
| Core and X | X gene | <CY5>CGTCTGTGCC<BHQ_2>TTCTCATCTG | 180, 181 |
| gene | | CCGGACCG<Spc_C3> | |
| Core and X | X gene | <5TEX_615>CGTCTGTGCC<BHQ_2>TTCTCA | 180, 181 |
| gene | | TCTGCCGGACCG<Spc_C3> | |

In Table 6, <D_LNA_T> refers to D-locked nucleic acid thymine, <D_LNA_G> refers to D-locked nucleic acid guanine, <BHQ_2> refers to Black Hole Quencher 2, <Spc_C3> refers to 3 carbon spacer, <CY5> refers to Cyanine5 fluorescent dye, <CY5.5> refers to Cyanine5 fluorescent dye variant, <5TEX 615> refers to Texas Red fluorescent dye, <5_FAM ABD> refers to fluorescein dye, HEG refers to hexaethylene glycol spacer, and <5_HEX_ABD> refers to hexachloro-fluorescein Dye. It will be appreciated that while specific dye, spacer and quencher molecules are assigned to the nucleic acid sequences in Table 6 (as well as throughout the present disclosure), it will be appreciated that substitutions may be possible. For example, one dye molecule may be substituted for another molecule without substantially affecting the utility of the corresponding nucleic acid sequence.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1.-4. (canceled)

5. A method to detect and quantify at least two different Hepatitis B Virus (HBV) target nucleic acids in a sample by Polymerase Chain Reaction (PCR), comprising,
providing the sample;
randomly distributing the sample into a plurality of equal sized and independent partitions;
performing in each partition a PCR assay with at least two forward and reverse primer sets for amplifying each of the HBV target nucleic acids, and with at least two probes, each probe labeled with a fluorescent dye that generates a different signal, for detecting each of the HBV target nucleic acids; and
measuring the amount of signal generated in each of the partitions to calculate the quantity of each of the at least two different HBV target nucleic in the sample.

6. The method of claim 5, wherein the PCR assay performed in each partition is a digital PCR (dPCR) assay.

7. The method of claim 5, wherein the at least two different HBV target nucleic acids are selected from the group consisting of Precore-mRNA 5′ end (non-pregenomic RNA), Core, X gene, Truncated RNA 3′ end (poly(A) junction), Precore/core, Full-length RNA 3′ end (poly(A) junction), Selected splice junctions, S gene (pre splice site), S gene (post splice site) and pregenomic RNA 5′ end.

8. The method of claim 5, wherein the dPCR assay is performed using the forward and reverse primer sets and probes that are specific for the HBV target nucleic acid that are selected from the oligonucleotides listed in Tables 4 and 6.

9. The method of claim 5, wherein the fluorescent dye on the labeled probe is selected from the group consisting of Atto-425, FAM, HEX, Texas Red, Cy5, and Cy5.5.

10. The method of claim 5, further comprising:
reducing unwanted amplification in the dPCR assay by the use of at least one blocker oligonucleotide having a non-extendable 3′ terminus and higher melting temperature (Tm) to template nucleic acid relative to the at least two forward and reverse primer sets and the at least two probes used in the dPCR assay.

11. The method of claim 10, wherein the unwanted amplification results from the amplification target regions being closely located.

12. The method of claim 10, wherein the unwanted amplification is the presence of DNA template where RNA is the target nucleic acid.

13. The method of claim 10, wherein the unwanted amplification is the presence of RNA splice variants in situations where the spliced RNA is the target and the unspliced RNA is inhibited.

14.-16. (canceled)

17. A method of reducing unwanted amplification in a multiplex Digital PCR (dPCR) assay by the use of a blocker oligonucleotide having a non-extendable 3′ terminus and higher melting temperature (Tm) to template nucleic acid relative to the primers and probes used in dPCR.

18. The method of claim 17, wherein the unwanted amplification results from the amplification target regions being closely located.

19. The method of claim 17, wherein the unwanted amplification is the presence of DNA template where RNA is the target nucleic acid.

20. The method of claim 17, wherein the unwanted amplification is the presence of RNA splice variants in situations where the spliced RNA is the target and the unspliced RNA is inhibited.

21. A kit for selectively detecting at least two targets in a nucleic acid, the kit comprising:
a first set of primers to produce a first amplification product if a first portion of a nucleic acid is present in the sample;
a second set of primers to produce a second amplification product if a second portion of the nucleic acid is present in the sample;
a blocker oligonucleotide complementary to the nucleic acid intermediate the first portion and the second portion;
a first detectable probe complementary to the first amplification product; and
a second detectable probe complementary to the second amplification product.

22. The kit of claim 21, wherein the blocker oligonucleotide is not extendable by a DNA polymerase.

23. The kit of claim 21, wherein the blocker oligonucleotide reduces or eliminates the production of an unwanted amplification product comprising the first target and the second target.

24. The method of claim 17, wherein the dPCR assay further comprises selectively detecting at least a first target and a second target in a sample, and wherein the primers comprise at least a first set of primers and a second set of primers, and wherein the probes comprise at least a first detectable probe and a second detectable probe.

25. The method of claim 24, wherein the dPCR assay further comprises:
performing an amplifying step comprising contacting the sample with:
the first set of primers to produce a first amplification product if a nucleic acid is present in the sample,
the second set of primers to produce a second amplification product if the nucleic acid is present in the sample, and
the blocker oligonucleotide;
performing a hybridizing step comprising contacting the first and second amplification products with at least the first detectable probe and the second detectable probe; and
detecting the presence or absence of the first and second amplification products,
wherein the presence of the first amplification product is indicative of the presence of the first target in the sample and wherein the absence of the first amplification product is indicative of the absence of the first target in the sample,
wherein the presence of the second amplification product is indicative of the presence of the second target in the sample and wherein the absence of the second amplification product is indicative of the absence of the second target in the sample, and
wherein the nucleic acid includes a continuous sequence comprising the first target, the second target, and an intermediate sequence positioned between the first target and the second target, and wherein the blocker oligonucleotide is complementary to at least a portion of the intermediate sequence.

26. The method of claim 24, wherein the blocker oligonucleotide is not extendable by a DNA polymerase.

27. The method of claim 25, wherein the blocker oligonucleotide reduces or eliminates the production of an unwanted amplification product comprising the first target and the second target.


SEQUENCE LISTING (181 sequences)

SEQ ID NO: 1 - DNA, 20 bp, synthetic construct
  source 1..20: mol_type = other DNA
gcaactttttcacctctgcc

SEQ ID NO: 2 - DNA, 20 bp, synthetic construct
  source 1..20: mol_type = other DNA
atacgggtcaatgtccatgc

SEQ ID NO: 3 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ccaagctgtgc

SEQ ID NO: 4 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ccaagctgtgc

SEQ ID NO: 5 - DNA, 26 bp, synthetic construct
  source 1..26: mol_type = other DNA
ttttttttttttttttttgaagctcc

SEQ ID NO: 6 - DNA, 20 bp, synthetic construct
  source 1..20: mol_type = other DNA
atacgggtcaatgtccatgt

SEQ ID NO: 7 - DNA, 20 bp, synthetic construct
  source 1..20: mol_type = other DNA
  modified_base 1: mod_base = OTHER; note = Fluorescein Dye modified nucleotide
  modified_base 20: mod_base = OTHER; note = Black hole quencher 2 modified nucleotide
ttcaagcctccaagctgtgc

SEQ ID NO: 8 - DNA, 20 bp, synthetic construct
  source 1..20: mol_type = other DNA
  modified_base 1: mod_base = OTHER; note = Fluorescein Dye modified nucleotide
  modified_base 20: mod_base = OTHER; note = Iowa Black FQ modified nucleotide
ttcaagcctccaagctgtgc

SEQ ID NO: 9 - DNA, 16 bp, synthetic construct
  source 1..16: mol_type = other DNA
gcgacgcggcgattga

SEQ ID NO: 10 - DNA, 18 bp, synthetic construct
  source 1..18: mol_type = other DNA
gaggcaggtcccctagaa

SEQ ID NO: 11 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tcgcctcgcagacg

SEQ ID NO: 12 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tcgcctcgcagacg

SEQ ID NO: 13 - DNA, 17 bp, synthetic construct
  source 1..17: mol_type = other DNA
  modified_base 1: mod_base = OTHER; note = ZEN quencher attached to a nucleotide sequence
  modified_base 17: mod_base = OTHER; note = Iowa Black FQ modified nucleotide
tcgcctcgcagacgaag

SEQ ID NO: 14 - DNA, 12 bp, synthetic construct
  source 1..12: mol_type = other DNA
  modified_base 1: mod_base = OTHER; note = ZEN quencher attached to a nucleotide sequence
  modified_base 12: mod_base = OTHER; note = Iowa Black FQ modified nucleotide
gcctcgcagacg

SEQ ID NO: 15 - DNA, 26 bp, synthetic construct
  source 1..26: mol_type = other DNA
  modified_base 1: mod_base = OTHER; note = Black hole quencher 2 attached to a nucleotide sequence
  modified_base 19: mod_base = OTHER; note = Phosphorylated nucleotide
agaactccctcgcctcgcagacgaag

SEQ ID NO: 16 - DNA, 18 bp, synthetic construct
  source 1..18: mol_type = other DNA
ggtgaagcgaagtgcaca

SEQ ID NO: 17 - DNA, 21 bp, synthetic construct
  source 1..21: mol_type = other DNA
acctctctttacgcggtctcc

SEQ ID NO: 18 - DNA, 17 bp, synthetic construct
  source 1..17: mol_type = other DNA
  modified_base 1: mod_base = OTHER; note = ZEN quencher attached to a nucleotide sequence
  modified_base 17: mod_base = OTHER; note = Iowa Black FQ modified nucleotide
tctcatctgccggaccg

SEQ ID NO: 19 - DNA, 17 bp, synthetic construct
  source 1..17: mol_type = other DNA
  modified_base 1: mod_base = OTHER; note = ZEN quencher attached to a nucleotide sequence
  modified_base 17: mod_base = OTHER; note = Iowa Black FQ modified nucleotide
tctcatctgccggaccg

SEQ ID NO: 20 - DNA, 19 bp, synthetic construct
  source 1..19: mol_type = other DNA
  modified_base 1: mod_base = OTHER; note = ZEN quencher attached to a nucleotide sequence
  modified_base 19: mod_base = OTHER; note = Iowa Black FQ modified nucleotide
cttctcatctgccggaccg

SEQ ID NO: 21 - DNA, 17 bp, synthetic construct
  source 1..17: mol_type = other DNA
  modified_base 1: mod_base = OTHER; note = ZEN quencher attached to a nucleotide sequence
  modified_base 17: mod_base = OTHER; note = Iowa Black FQ modified nucleotide
cttctcatctgccggac

SEQ ID NO: 22 - DNA, 28 bp, synthetic construct
  source 1..28: mol_type = other DNA
  modified_base 1: mod_base = OTHER; note = Black hole quencher 2 attached to a nucleotide sequence
  modified_base 21: mod_base = OTHER; note = Phosphorylated nucleotide
cgtctgtgccttctcatctgccggaccg

SEQ ID NO: 23 - DNA, 23 bp, synthetic construct
  source 1..23: mol_type = other DNA
  modified_base 1: mod_base = OTHER; note = Fluorescein Dye modified nucleotide
  modified_base 23: mod_base = OTHER; note = Iowa Black FQ modified nucleotide
agaactccctcgcctcgcagacg

SEQ ID NO: 24 - DNA, 23 bp, synthetic construct
  source 1..23: mol_type = other DNA
  modified_base 1: mod_base = OTHER; note = Fluorescein Dye modified nucleotide
  modified_base 23: mod_base = OTHER; note = Iowa Black FQ modified nucleotide
agaactccctcgcctcgcagacg

SEQ ID NO: 25 - DNA, 23 bp, synthetic construct
  source 1..23: mol_type = other DNA
  modified_base 1: mod_base = OTHER; note = Hexachloro-Fluorescein Dye modified nucleotide
  modified_base 23: mod_base = OTHER; note = Iowa Black FQ modified nucleotide
agaactccctcgcctcgcagacg

SEQ ID NO: 26 - DNA, 26 bp, synthetic construct
  source 1..26: mol_type = other DNA
  modified_base 1: mod_base = OTHER; note = Hexachloro-Fluorescein Dye modified nucleotide
  modified_base 26: mod_base = OTHER; note = Iowa Black FQ modified nucleotide
tctgtgccttctcatctgccggaccg

SEQ ID NO: 27 - DNA, 20 bp, synthetic construct
  source 1..20: mol_type = other DNA
atacgggtcaatgtccatgc

SEQ ID NO: 28 - DNA, 20 bp, synthetic construct
  source 1..20: mol_type = other DNA
gcaactttttcacctctgcc

SEQ ID NO: 29 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ccaagctgtgc

SEQ ID NO: 30 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ccaagctgtgc

SEQ ID NO: 31 - DNA, 26 bp, synthetic construct
  source 1..26: mol_type = other DNA
ttttttttttttttttttgaagctcc

SEQ ID NO: 32 - DNA, 26 bp, synthetic construct
  source 1..26: mol_type = other DNA
ttttttttttttttttttgtagctcc

SEQ ID NO: 33 - DNA, 23 bp, synthetic construct
  source 1..23: mol_type = other DNA
ttttttttttttttttttgaagc

SEQ ID NO: 34 - DNA, 23 bp, synthetic construct
  source 1..23: mol_type = other DNA
ttttttttttttttttttgtagc

SEQ ID NO: 35 - DNA, 25 bp, synthetic construct
  source 1..25: mol_type = other DNA
ttttttttttttttttttgaagctc

SEQ ID NO: 36 - DNA, 25 bp, synthetic construct
  source 1..25: mol_type = other DNA
ttttttttttttttttttgtagctc

SEQ ID NO: 37 - DNA, 26 bp, synthetic construct
  source 1..26: mol_type = other DNA
ttttttttttttttttttgaagctcc

SEQ ID NO: 38 - DNA, 26 bp, synthetic construct
  source 1..26: mol_type = other DNA
ttttttttttttttttttgtagctcc

SEQ ID NO: 39 - DNA, 24 bp, synthetic construct
  source 1..24: mol_type = other DNA
ttttttttttttttgaagctccaa

SEQ ID NO: 40 - DNA, 24 bp, synthetic construct
  source 1..24: mol_type = other DNA
ttttttttttttttgtagctccaa

SEQ ID NO: 41 - DNA, 28 bp, synthetic construct
  source 1..28: mol_type = other DNA
ttttttttttttttttttgaagctccaa

SEQ ID NO: 42 - DNA, 28 bp, synthetic construct
  source 1..28: mol_type = other DNA
ttttttttttttttttttgtagctccaa

SEQ ID NO: 43 - DNA, 49 bp, synthetic construct
  source 1..49: mol_type = other DNA
  modified_base 27: mod_base = OTHER; note = Locked thymidine (beta D LNA)
  modified_base 31: mod_base = OTHER; note = Locked thymidine (beta D LNA)
  modified_base 35: mod_base = OTHER; note = Locked thymidine (beta D LNA)
  modified_base 39: mod_base = OTHER; note = Locked thymidine (beta D LNA)
  modified_base 41: mod_base = OTHER; note = Locked thymidine (beta D LNA)
gatcaacgtgtcaccgcctattctatttttttttttttttttgaagctc

SEQ ID NO: 44 - DNA, 25 bp, synthetic construct
  source 1..25: mol_type = other DNA
catgcaactttttcacctctgccta

SEQ ID NO: 45 - DNA, 20 bp, synthetic construct
  source 1..20: mol_type = other DNA
  modified_base 1: mod_base = OTHER; note = Fluorescein Dye modified nucleotide
  modified_base 20: mod_base = OTHER; note = Iowa Black FQ modified nucleotide
ttcaagcctccaagctgtgc

SEQ ID NO: 46 - DNA, 20 bp, synthetic construct
  source 1..20: mol_type = other DNA
  modified_base 1: mod_base = OTHER; note = Fluorescein Dye modified nucleotide
  modified_base 20: mod_base = OTHER; note = Black hole quencher 2 modified nucleotide
ttcaagcctccaagctgtgc

SEQ ID NO: 47 - DNA, 18 bp, synthetic construct
  source 1..18: mol_type = other DNA
ggtctgttcaccagcacc

SEQ ID NO: 48 - DNA, 23 bp, synthetic construct
  source 1..23: mol_type = other DNA
gaaggaaagaagtcagaaggcaa

SEQ ID NO: 49 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ggacattgacc

SEQ ID NO: 50 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
ggacattgacc

SEQ ID NO: 51 - DNA, 18 bp, synthetic construct
  source 1..18: mol_type = other DNA
ggtctgcgcaccagcacc

SEQ ID NO: 52 - DNA, 25 bp, synthetic construct
  source 1..25: mol_type = other DNA
ggaaagaagtcagaaggcaaaaacg

SEQ ID NO: 53 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tggacattgacccg

SEQ ID NO: 54 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tggacattgacccg

SEQ ID NO: 55 - DNA, 20 bp, synthetic construct
  source 1..20: mol_type = other DNA
  modified_base 1: mod_base = OTHER; note = Hexachloro-Fluorescein Dye modified nucleotide
  modified_base 20: mod_base = OTHER; note = Iowa Black FQ modified nucleotide
ttggggcatggacattgacc

SEQ ID NO: 56 - DNA, 20 bp, synthetic construct
  source 1..20: mol_type = other DNA
  modified_base 1: mod_base = OTHER; note = Fluorescein Dye modified nucleotide
  modified_base 20: mod_base = OTHER; note = Iowa Black FQ modified nucleotide
ttggggcatggacattgacc

SEQ ID NO: 57 - DNA, 23 bp, synthetic construct
  source 1..23: mol_type = other DNA
  modified_base 23: mod_base = OTHER; note = tert-butyl-benzyl-deoxy-cytosine
actcttggactctcagcaatgtc

SEQ ID NO: 58 - DNA, 10 bp, synthetic construct
  source 1..10: mol_type = other DNA
  modified_base 1: mod_base = OTHER; note = Fluorescein Dye modified nucleotide
  modified_base 10: mod_base = OTHER; note = ZEN quencher attached to a nucleotide sequence
accgaccttg

SEQ ID NO: 59 - DNA, 10 bp, synthetic construct
  source 1..10: mol_type = other DNA
  modified_base 1: mod_base = OTHER; note = Hexachloro-Fluorescein Dye modified nucleotide
  modified_base 10: mod_base = OTHER; note = ZEN quencher attached to a nucleotide sequence
accgaccttg

SEQ ID NO: 60 - DNA, 25 bp, synthetic construct
  source 1..25: mol_type = other DNA
ttttttttttttttttttgctggtg

SEQ ID NO: 61 - DNA, 35 bp, synthetic construct
  source 1..35: mol_type = other DNA
  modified_base 35: mod_base = OTHER; note = C3 Spacer modified nucleotide
gttgcatggtgctggtgaacagaccaatttatgcc

SEQ ID NO: 62 - DNA, 50 bp, synthetic construct
  source 1..50: mol_type = other DNA
  modified_base 50: mod_base = OTHER; note = C3 Spacer modified nucleotide
taggcagaggtgaaaaagttgcatggtgctggtgaacagaccaatttatg

SEQ ID NO: 63 - DNA, 23 bp, synthetic construct
  source 1..23: mol_type = other DNA
  modified_base 23: mod_base = OTHER; note = tert-butyl-benzyl-deoxy-cytosine
actcttggactctctgtaatgtc

SEQ ID NO: 64 - DNA, 34 bp, synthetic construct
  source 1..34: mol_type = other DNA
  modified_base 1: mod_base = OTHER; note = Black hole quencher 2 attached to a nucleotide sequence
  modified_base 34: mod_base = OTHER; note = Phosphorylated nucleotide
ttgaggcatacttcaaagactgtgtgtttaaaga

SEQ ID NO: 65 - DNA, 34 bp, synthetic construct
  source 1..34: mol_type = other DNA
  modified_base 1: mod_base = OTHER; note = Black hole quencher 2 attached to a nucleotide sequence
  modified_base 34: mod_base = OTHER; note = Phosphorylated nucleotide
tcgaagaatacatcaaagactgtgtatttaagga

SEQ ID NO: 66 - DNA, 28 bp, synthetic construct
  source 1..28: mol_type = other DNA
ttttttttttttttttttgctggtgaac

SEQ ID NO: 67 - DNA, 28 bp, synthetic construct
  source 1..28: mol_type = other DNA
ttttttttttttttttttgctggtgcgc

SEQ ID NO: 68 - DNA, 32 bp, synthetic construct
  source 1..32: mol_type = other DNA
ttttttttttttttttttgctggtgaacagac

SEQ ID NO: 69 - DNA, 23 bp, synthetic construct
  source 1..23: mol_type = other DNA
ttttttttttttttttttgctgg

SEQ ID NO: 70 - DNA, 10 bp, synthetic construct
  source 1..10: mol_type = other DNA
  modified_base 1: mod_base = OTHER; note = Fluorescein Dye modified nucleotide
  modified_base 10: mod_base = OTHER; note = ZEN quencher attached to a nucleotide sequence
accttgaggc

SEQ ID NO: 71 - DNA, 10 bp, synthetic construct
  source 1..10: mol_type = other DNA
  modified_base 1: mod_base = OTHER; note = Hexachloro-Fluorescein Dye modified nucleotide
  modified_base 10: mod_base = OTHER; note = ZEN quencher attached to a nucleotide sequence
accttgaggc

SEQ ID NO: 72 - DNA, 10 bp, synthetic construct
  source 1..10: mol_type = other DNA
  modified_base 1: mod_base = OTHER; note = Fluorescein Dye modified nucleotide
  modified_base 10: mod_base = OTHER; note = ZEN quencher attached to a nucleotide sequence
accgaccttg

SEQ ID NO: 73 - DNA, 10 bp, synthetic construct
  source 1..10: mol_type = other DNA
  modified_base 1: mod_base = OTHER; note = Hexachloro-Fluorescein Dye modified nucleotide
  modified_base 10: mod_base = OTHER; note = ZEN quencher attached to a nucleotide sequence
accgaccttg

SEQ ID NO: 74 - DNA, 20 bp, synthetic construct
  source 1..20: mol_type = other DNA
gagactcgactccacaacca

SEQ ID NO: 75 - DNA, 51 bp, synthetic construct
  source 1..51: mol_type = other DNA
gagactcgactccacaaccatttttttttttttttttgctggtgaacagac

SEQ ID NO: 76 - DNA, 20 bp, synthetic construct
  source 1..20: mol_type = other DNA
ccgaccttgaggcatacttc

SEQ ID NO: 77 - DNA, 26 bp, synthetic construct
  source 1..26: mol_type = other DNA
  modified_base 1: mod_base = OTHER; note = Fluorescein Dye modified nucleotide
  modified_base 26: mod_base = OTHER; note = Black hole quencher 1 modified nucleotide
cagaccaatttatgcctacagcctcc

SEQ ID NO: 78 - DNA, 22 bp, synthetic construct
  source 1..22: mol_type = other DNA
  modified_base 1: mod_base = OTHER; note = 5-propynyl deoxyuridine
  modified_base 9: mod_base = OTHER; note = 5-propynyl deoxyuridine
  modified_base 11: mod_base = OTHER; note = 5-methyl cytidine (DNA)
  modified_base 13: mod_base = OTHER; note = 5-propynyl deoxyuridine
  modified_base 14: mod_base = OTHER; note = 5-methyl cytidine (DNA)
  modified_base 20: mod_base = OTHER; note = 5-methyl cytidine (DNA)
ttgaggcatacttcaaagactg

SEQ ID NO: 79 - DNA, 10 bp, synthetic construct
  source 1..10: mol_type = other DNA
  modified_base 1: mod_base = OTHER; note = Hexachloro-Fluorescein Dye modified nucleotide
  modified_base 3: mod_base = OTHER; note = 5-propynyl deoxyuridine
  modified_base 6: mod_base = OTHER; note = 5-propynyl deoxyuridine
  modified_base 10: mod_base = OTHER; note = Black hole quencher 2 attached to a nucleotide sequence
tgtactagga

SEQ ID NO: 80 - DNA, 19 bp, synthetic construct
  source 1..19: mol_type = other DNA
cgtcgcagaagatctcaat

SEQ ID NO: 81 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
ctcaatgaacatca

SEQ ID NO: 82 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
caaaattcgca

SEQ ID NO: 83 - DNA, 19 bp, synthetic construct
  source 1..19: mol_type = other DNA
gttccttgagcaggaatcg

SEQ ID NO: 84 - DNA, 20 bp, synthetic construct
  source 1..20: mol_type = other DNA
ttagagtctccggaacattg

SEQ ID NO: 85 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
tcagacgacga

SEQ ID NO: 86 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
tcagacgacga

SEQ ID NO: 87 - DNA, 21 bp, synthetic construct
  source 1..21: mol_type = other DNA
cgagggagttcttcttctagg

SEQ ID NO: 88 - DNA, 19 bp, synthetic construct
  source 1..19: mol_type = other DNA
ttccttgagcaggaatcgt

SEQ ID NO: 89 - DNA, 21 bp, synthetic construct
  source 1..21: mol_type = other DNA
cattgttcacctcaccataca

SEQ ID NO: 90 - DNA, 13 bp, synthetic construct
  source 1..13: mol_type = other DNA
  modified_base 1: mod_base = OTHER; note = ZEN quencher attached to a nucleotide sequence
  modified_base 13: mod_base = OTHER; note = Iowa Black FQ modified nucleotide
atcaactaccagc

SEQ ID NO: 91 - DNA, 13 bp, synthetic construct
  source 1..13: mol_type = other DNA
  modified_base 1: mod_base = OTHER; note = ZEN quencher attached to a nucleotide sequence
  modified_base 13: mod_base = OTHER; note = Iowa Black FQ modified nucleotide
atcaactaccagc

SEQ ID NO: 92 - DNA, 20 bp, synthetic construct
  source 1..20: mol_type = other DNA
gttccttgagcaggaatcgt

SEQ ID NO: 93 - DNA, 20 bp, synthetic construct
  source 1..20: mol_type = other DNA
cattgttcacctcaccatac

SEQ ID NO: 94-95 - skipped (2 entries)

SEQ ID NO: 96 - DNA, 21 bp, synthetic construct
  source 1..21: mol_type = other DNA
cgagggagttcttcttctagg

SEQ ID NO: 97 - DNA, 18 bp, synthetic construct
  source 1..18: mol_type = other DNA
gttccttgagcaggaatc

SEQ ID NO: 98 - DNA, 18 bp, synthetic construct
  source 1..18: mol_type = other DNA
ttccttgagcaggaatcg

SEQ ID NO: 99 - DNA, 18 bp, synthetic construct
  source 1..18: mol_type = other DNA
agcactcaggcaagctat

SEQ ID NO: 100 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
caactaccagc

SEQ ID NO: 101 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
caactaccagc

SEQ ID NO: 102 - DNA, 20 bp, synthetic construct
  source 1..20: mol_type = other DNA
gttccttgagcaggaatcgt

SEQ ID NO: 103 - DNA, 17 bp, synthetic construct
  source 1..17: mol_type = other DNA
cgcgtcgcagaagatct

SEQ ID NO: 104 - DNA, 22 bp, synthetic construct
  source 1..22: mol_type = other DNA
atgggaatacaggtgcaatttc

SEQ ID NO: 105 - DNA, 18 bp, synthetic construct
  source 1..18: mol_type = other DNA
ccgcgtcgcagaagatct

SEQ ID NO: 106 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tctcaatgaacatc

SEQ ID NO: 107 - DNA, 14 bp, synthetic construct
  source 1..14: mol_type = other DNA
  modified_base 1: mod_base = OTHER; note = ZEN quencher attached to a nucleotide sequence
  modified_base 14: mod_base = OTHER; note = Iowa Black FQ modified nucleotide
tctcaatgaacatc

SEQ ID NO: 108 - DNA, 23 bp, synthetic construct
  source 1..23: mol_type = other DNA
atgggaatacaggtgcaatttcc

SEQ ID NO: 109 - DNA, 20 bp, synthetic construct
  source 1..20: mol_type = other DNA
tagactcgtggtgcagttct

SEQ ID NO: 110 - DNA, 19 bp, synthetic construct
  source 1..19: mol_type = other DNA
atagcagcaggatgaagcg

SEQ ID NO: 111 - DNA, 25 bp, synthetic construct
  source 1..25: mol_type = other DNA
tctagactcgtggtgcagttctctc

SEQ ID NO: 112 - DNA, 13 bp, synthetic construct
  source 1..13: mol_type = other DNA
  modified_base 1: mod_base = OTHER; note = ZEN quencher attached to a nucleotide sequence
  modified_base 13: mod_base = OTHER; note = Iowa Black FQ modified nucleotide
ccaacctccaatc

SEQ ID NO: 113 - DNA, 13 bp, synthetic construct
  source 1..13: mol_type = other DNA
  modified_base 1: mod_base = OTHER; note = ZEN quencher attached to a nucleotide sequence
  modified_base 13: mod_base = OTHER; note = Iowa Black FQ modified nucleotide
ccaacctccaatc

SEQ ID NO: 114 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
caaaattcgca

SEQ ID NO: 115 - DNA, 11 bp, synthetic construct
  source 1..11: mol_type = other DNA
  modified_base 1: mod_base = OTHER; note = ZEN quencher attached to a nucleotide sequence
  modified_base 11: mod_base = OTHER; note = Iowa Black FQ modified nucleotide
caaaattcgca

SEQ ID NO: 116 - DNA, 23 bp, synthetic construct
  source 1..23: mol_type = other DNA
catagcagcaggatgaagcggaa

SEQ ID NO: 117 - DNA, 22 bp, synthetic construct
  source 1..22: mol_type = other DNA
gcacctgtattcccatcccatc

SEQ ID NO: 118 - DNA, 20 bp, synthetic construct
  source 1..20: mol_type = other DNA
  modified_base 1: mod_base = OTHER; note = ZEN quencher attached to a nucleotide sequence
  modified_base 20: mod_base = OTHER; note = Iowa Black FQ modified nucleotide
ctgaggcccactcccatagg

SEQ ID NO: 119 - DNA, 20 bp, synthetic construct
  source 1..20: mol_type = other DNA
  modified_base 1: mod_base = OTHER; note = ZEN quencher attached to a nucleotide sequence
  modified_base 20: mod_base = OTHER; note = Iowa Black FQ modified nucleotide
ctgaggcccactcccatagg

SEQ ID NO: 120 - DNA, 25 bp, synthetic construct
  source 1..25: mol_type = other DNA
caaatggcactagtaaactgagcca

SEQ ID NO: 121 - DNA, 26 bp, synthetic construct
  source 1..26: mol_type = other DNA
  modified_base 3: mod_base = OTHER; note = 7-deaza deoxyguanine
cggggtttttcttgttgacaagaatc

SEQ ID NO: 122 - DNA, 26 bp, synthetic construct
  source 1..26: mol_type = other DNA
  modified_base 4: mod_base = OTHER; note = 7-deaza deoxyguanine
cggggtttttcttgttgacaagaatc

SEQ ID NO: 123 - DNA, 27 bp, synthetic construct
  source 1..27: mol_type = other DNA
  modified_base 1: mod_base = OTHER; note = ZEN quencher attached to a nucleotide sequence
  modified_base 27: mod_base = OTHER; note = Iowa Black FQ modified nucleotide
cgagtctagactctgtggtattgtgag

SEQ ID NO: 124 - DNA, 27 bp, synthetic construct
  source 1..27: mol_type = other DNA
  modified_base 1: mod_base = OTHER; note = ZEN quencher attached to a nucleotide sequence
  modified_base 27: mod_base = OTHER; note = Iowa Black FQ modified nucleotide
cgagtctagactctgtggtattgtgag

SEQ ID NO: 125 - DNA, 18 bp, synthetic construct
  source 1..18: mol_type = other DNA
  modified_base 8: mod_base = OTHER; note = 7-deaza deoxyguanine
gaggttggggactgcgaa

SEQ ID NO: 126 - DNA, 18 bp, synthetic construct
  source 1..18: mol_type = other DNA
  modified_base 9: mod_base = OTHER; note = 7-deaza deoxyguanine
gaggttggggactgcgaa

SEQ ID NO: 127 - DNA, 21 bp, synthetic construct
  source 1..21: mol_type = other DNA
  modified_base 1: mod_base = OTHER; note = Black hole quencher 2 attached to a nucleotide sequence
  modified_base 21: mod_base = OTHER; note = C3 Spacer modified nucleotide
actgaggcccactcccatagg

SEQ ID NO: 128 - DNA, 21 bp, synthetic construct
  source 1..21: mol_type = other DNA
  modified_base 1: mod_base = OTHER; note = Black hole quencher 2 attached to a nucleotide sequence
  modified_base 21: mod_base = OTHER; note = C3 Spacer modified nucleotide
actgaggcccactcccatagg

SEQ ID NO: 129 - DNA, 26 bp, synthetic construct
  source 1..26: mol_type = other DNA
ctgaacaaatggcactagtaaactga

SEQ ID NO: 130 - DNA, 26 bp, synthetic construct
  source 1..26: mol_type = other DNA
accactgaacaaatggcactagtaaa

SEQ ID NO: 131 - DNA, 26 bp, synthetic construct
  source 1..26: mol_type = other DNA
  modified_base 3: mod_base = OTHER; note = 7-deaza deoxyguanine
cggggtttttcttgttgacaagaatc

SEQ ID NO: 132 - DNA, 26 bp, synthetic construct
  source 1..26: mol_type = other DNA
  modified_base 2: mod_base = OTHER; note = 7-deaza deoxyguanine
ggggtttttcttgttgacaagaatcc

SEQ ID NO: 133 - DNA, 25 bp, synthetic construct
  source 1..25: mol_type = other DNA
  modified_base 3: mod_base = OTHER; note = 7-deaza deoxyguanine
gggtttttcttgttgacaagaatcc

SEQ ID NO: 134 - DNA, 28 bp, synthetic construct
  source 1..28: mol_type = other DNA
  modified_base 1: mod_base = OTHER; note = Black hole quencher 2 attached to a nucleotide sequence
  modified_base 28: mod_base = OTHER; note = C3 Spacer modified nucleotide
acgagtctagactctgtggtattgtgag

SEQ ID NO: 135 - DNA, 28 bp, synthetic construct
  source 1..28: mol_type = other DNA
  modified_base 1: mod_base = OTHER; note = Black hole quencher 2 attached to a nucleotide sequence
  modified_base 28: mod_base = OTHER; note = C3 Spacer modified nucleotide
acgagtctagactctgtggtattgtgag

SEQ ID NO: 136 - DNA, 17 bp, synthetic construct
  source 1..17: mol_type = other DNA
  modified_base 10: mod_base = OTHER; note = 7-deaza deoxyguanine
tggaggttggggactgc

SEQ ID NO: 137 - DNA, 17 bp, synthetic construct
  source 1..17: mol_type = other DNA
  modified_base 11: mod_base = OTHER; note = 7-deaza deoxyguanine
tggaggttggggactgc

SEQ ID NO: 138 - DNA, 31 bp, synthetic construct
  source 1..31: mol_type = other DNA
  modified_base 1: mod_base = OTHER; note = ZEN quencher attached to a nucleotide sequence
  modified_base 31: mod_base = OTHER; note = Iowa Black FQ modified nucleotide
aggcatacttcaaagactgtgtgtttaaaga

SEQ ID NO: 139 - DNA, 31 bp, synthetic construct; identical to SEQ ID NO: 138
  source 1..31: mol_type = other DNA
  modified_base 1: mod_base = OTHER; note = ZEN quencher attached to a nucleotide sequence
  modified_base 31: mod_base = OTHER; note = Iowa Black FQ modified nucleotide

SEQ ID NO: 140 - DNA, 23 bp, synthetic construct
  source 1..23: mol_type = other DNA
  modified_base 1: mod_base = OTHER; note = ZEN quencher attached to a nucleotide sequence
  modified_base 23: mod_base = OTHER; note = Iowa Black FQ modified nucleotide
atacttcaaagactgtgtgttta

SEQ ID NO: 141 - DNA, 23 bp, synthetic construct
  source 1..23: mol_type = other DNA
  modified_base 1: mod_base = OTHER; note = ZEN quencher attached to a nucleotide sequence
  modified_base 23: mod_base = OTHER; note = Iowa Black FQ modified nucleotide
atacttcaaagactgtgtgttta

SEQ ID NO: 142 - DNA, 27 bp, synthetic construct
  source 1..27: mol_type = other DNA
  modified_base 1: mod_base = OTHER; note = ZEN quencher attached to a nucleotide sequence
  modified_base 27: mod_base = OTHER; note = Iowa Black FQ modified nucleotide
aggcatacttcaaagactgtgtgttta

SEQ ID NO: 143 - DNA, 27 bp, synthetic construct
  source 1..27: mol_type = other DNA
  modified_base 1: mod_base = OTHER; note = ZEN quencher attached to a nucleotide sequence
  modified_base 27: mod_base = OTHER; note = Iowa Black FQ modified nucleotide
aggcatacttcaaagactgtgtgttta

SEQ ID NO: 144 - DNA, 18 bp, synthetic construct
  source 1..18: mol_type = other DNA
  modified_base 1: mod_base = OTHER; note = Black hole quencher 2 attached to a nucleotide sequence
  modified_base 3: mod_base = OTHER; note = 5-methyl cytidine (DNA)
  modified_base 10: mod_base = OTHER; note = 5-methyl cytidine (DNA)
  modified_base 18: mod_base = OTHER; note = Phosphorylated nucleotide
ggctgtaggcataaattg

SEQ ID NO: 145 - DNA, 24 bp, synthetic construct
  source 1..24: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
tttttttttttttttttgaagctc

SEQ ID NO: 146 - DNA, 24 bp, synthetic construct
  source 1..24: mol_type = other DNA
  modified_base 2: mod_base = OTHER; note = D-LNA
  modified_base 4: mod_base = OTHER; note = D-LNA
  modified_base 6: mod_base = OTHER; note = D-LNA
  modified_base 8: mod_base = OTHER; note = D-LNA
  modified_base 10: mod_base = OTHER; note = D-LNA
  modified_base 12: mod_base = OTHER; note = D-LNA
  modified_base 14: mod_base = OTHER; note = D-LNA
  modified_base 16: mod_base = OTHER; note = D-LNA
tttttttttttttttttgaagctc

SEQ ID NO: 147 - DNA, 43 bp, synthetic construct
  source 1..43: mol_type = other DNA
  modified_base 21: mod_base = OTHER; note = D-LNA
  modified_base 25: mod_base = OTHER; note = D-LNA
  modified_base 29: mod_base = OTHER; note = D-LNA
  modified_base 33: mod_base = OTHER; note = D-LNA
  modified_base 35: mod_base = OTHER; note = D-LNA
gatcaacgtgtcaccgccttttttttttttttttttgaagctc

SEQ ID NO: 148 - DNA, 43 bp, synthetic construct; identical to SEQ ID NO: 147
  source 1..43: mol_type = other DNA
  modified_base 21: mod_base = OTHER; note = D-LNA
  modified_base 23: mod_base = OTHER; note = D-LNA
  modified_base 25: mod_base = OTHER; note = D-LNA
  modified_base 27: mod_base = OTHER; note = D-LNA
  modified_base 29: mod_base = OTHER; note = D-LNA
  modified_base 31: mod_base = OTHER; note = D-LNA
  modified_base 33: mod_base = OTHER; note = D-LNA
  modified_base 35: mod_base = OTHER; note = D-LNA

SEQ ID NO: 149 - DNA, 49 bp, synthetic construct; identical to SEQ ID NO: 43
  source 1..49: mol_type = other DNA
  modified_base 27: mod_base = OTHER; note = D-LNA
  modified_base 31: mod_base = OTHER; note = D-LNA
  modified_base 35: mod_base = OTHER; note = v
  modified_base 39: mod_base = OTHER; note = D-LNA
  modified_base 41: mod_base = OTHER; note = D-LNA

SEQ ID NO: 150 - DNA, 24 bp, synthetic construct
  source 1..24: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
  modified_base 21: mod_base = OTHER; note = D-LNA
tttttttttttttttttgaagctc

SEQ ID NO: 151 - DNA, 25 bp, synthetic construct
  source 1..25: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
tttttttttttttttttgaagctcc

SEQ ID NO: 152 - DNA, 25 bp, synthetic construct
  source 1..25: mol_type = other DNA
  modified_base 2: mod_base = OTHER; note = D-LNA
  modified_base 4: mod_base = OTHER; note = D-LNA
  modified_base 6: mod_base = OTHER; note = D-LNA
  modified_base 8: mod_base = OTHER; note = D-LNA
  modified_base 10: mod_base = OTHER; note = D-LNA
  modified_base 12: mod_base = OTHER; note = D-LNA
  modified_base 14: mod_base = OTHER; note = D-LNA
  modified_base 16: mod_base = OTHER; note = D-LNA
tttttttttttttttttgaagctcc

SEQ ID NO: 153 - DNA, 44 bp, synthetic construct
  source 1..44: mol_type = other DNA
  modified_base 21: mod_base = OTHER; note = D-LNA
  modified_base 25: mod_base = OTHER; note = D-LNA
  modified_base 29: mod_base = OTHER; note = D-LNA
  modified_base 33: mod_base = OTHER; note = D-LNA
  modified_base 35: mod_base = OTHER; note = D-LNA
gatcaacgtgtcaccgccttttttttttttttttttgaagctcc

SEQ ID NO: 154 - DNA, 44 bp, synthetic construct; identical to SEQ ID NO: 153
  source 1..44: mol_type = other DNA
  modified_base 21: mod_base = OTHER; note = D-LNA
  modified_base 23: mod_base = OTHER; note = D-LNA
  modified_base 25: mod_base = OTHER; note = D-LNA
  modified_base 27: mod_base = OTHER; note = D-LNA
  modified_base 29: mod_base = OTHER; note = D-LNA
  modified_base 31: mod_base = OTHER; note = D-LNA
  modified_base 33: mod_base = OTHER; note = D-LNA
  modified_base 35: mod_base = OTHER; note = D-LNA

SEQ ID NO: 155 - DNA, 49 bp, synthetic construct
  source 1..49: mol_type = other DNA
  modified_base 26: mod_base = OTHER; note = D-LNA
  modified_base 30: mod_base = OTHER; note = D-LNA
  modified_base 34: mod_base = OTHER; note = D-LNA
  modified_base 38: mod_base = OTHER; note = D-LNA
  modified_base 40: mod_base = OTHER; note = D-LNA
gatcaacgtgtcaccgcctattctattttttttttttttttgaagctcc

SEQ ID NO: 156 - DNA, 25 bp, synthetic construct
  source 1..25: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
  modified_base 21: mod_base = OTHER; note = D-LNA
tttttttttttttttttgaagctcc

SEQ ID NO: 157 - DNA, 26 bp, synthetic construct
  source 1..26: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
tttttttttttttttttgaagctcca

SEQ ID NO: 158 - DNA, 26 bp, synthetic construct
  source 1..26: mol_type = other DNA
  modified_base 2: mod_base = OTHER; note = D-LNA
  modified_base 4: mod_base = OTHER; note = D-LNA
  modified_base 6: mod_base = OTHER; note = D-LNA
  modified_base 8: mod_base = OTHER; note = D-LNA
  modified_base 10: mod_base = OTHER; note = D-LNA
  modified_base 12: mod_base = OTHER; note = D-LNA
  modified_base 14: mod_base = OTHER; note = D-LNA
  modified_base 16: mod_base = OTHER; note = D-LNA
tttttttttttttttttgaagctcca

SEQ ID NO: 159 - DNA, 45 bp, synthetic construct
  source 1..45: mol_type = other DNA
  modified_base 21: mod_base = OTHER; note = D-LNA
  modified_base 25: mod_base = OTHER; note = D-LNA
  modified_base 29: mod_base = OTHER; note = D-LNA
  modified_base 33: mod_base = OTHER; note = D-LNA
  modified_base 35: mod_base = OTHER; note = D-LNA
gatcaacgtgtcaccgccttttttttttttttttttgaagctcca

SEQ ID NO: 160 - DNA, 45 bp, synthetic construct; identical to SEQ ID NO: 159
  source 1..45: mol_type = other DNA
  modified_base 21: mod_base = OTHER; note = D-LNA
  modified_base 23: mod_base = OTHER; note = D-LNA
  modified_base 25: mod_base = OTHER; note = D-LNA
  modified_base 27: mod_base = OTHER; note = D-LNA
  modified_base 29: mod_base = OTHER; note = D-LNA
  modified_base 31: mod_base = OTHER; note = D-LNA
  modified_base 33: mod_base = OTHER; note = D-LNA
  modified_base 35: mod_base = OTHER; note = D-LNA

SEQ ID NO: 161 - DNA, 49 bp, synthetic construct
  source 1..49: mol_type = other DNA
  modified_base 26: mod_base = OTHER; note = D-LNA
  modified_base 29: mod_base = OTHER; note = D-LNA
  modified_base 33: mod_base = OTHER; note = D-LNA
  modified_base 37: mod_base = OTHER; note = D-LNA
  modified_base 39: mod_base = OTHER; note = D-LNA
gatcaacgtgtcaccgcctattctatttttttttttttttgaagctcca

SEQ ID NO: 162 - DNA, 26 bp, synthetic construct
  source 1..26: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
  modified_base 21: mod_base = OTHER; note = D-LNA
tttttttttttttttttgaagctcca

SEQ ID NO: 163 - DNA, 27 bp, synthetic construct
  source 1..27: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
tttttttttttttttttgaagctccaa

SEQ ID NO: 164 - DNA, 27 bp, synthetic construct
  source 1..27: mol_type = other DNA
  modified_base 2: mod_base = OTHER; note = D-LNA
  modified_base 4: mod_base = OTHER; note = D-LNA
  modified_base 6: mod_base = OTHER; note = D-LNA
  modified_base 8: mod_base = OTHER; note = D-LNA
  modified_base 10: mod_base = OTHER; note = D-LNA
  modified_base 12: mod_base = OTHER; note = D-LNA
  modified_base 16: mod_base = OTHER; note = D-LNA
tttttttttttttttttgaagctccaa

SEQ ID NO: 165 - DNA, 46 bp, synthetic construct
  source 1..46: mol_type = other DNA
  modified_base 21: mod_base = OTHER; note = D-LNA
  modified_base 25: mod_base = OTHER; note = D-LNA
  modified_base 29: mod_base = OTHER; note = D-LNA
  modified_base 33: mod_base = OTHER; note = D-LNA
  modified_base 35: mod_base = OTHER; note = D-LNA
gatcaacgtgtcaccgccttttttttttttttttttgaagctccaa

SEQ ID NO: 166 - DNA, 49 bp, synthetic construct
  source 1..49: mol_type = other DNA
  modified_base 26: mod_base = OTHER; note = D-LNA
  modified_base 29: mod_base = OTHER; note = D-LNA
  modified_base 32: mod_base = OTHER; note = D-LNA
  modified_base 36: mod_base = OTHER; note = D-LNA
  modified_base 38: mod_base = OTHER; note = D-LNA
gatcaacgtgtcaccgcctattctattttttttttttttgaagctccaa

SEQ ID NO: 167 - DNA, 46 bp, synthetic construct; identical to SEQ ID NO: 165
  source 1..46: mol_type = other DNA
  modified_base 21: mod_base = OTHER; note = D-LNA
  modified_base 23: mod_base = OTHER; note = D-LNA
  modified_base 25: mod_base = OTHER; note = D-LNA
  modified_base 27: mod_base = OTHER; note = D-LNA
  modified_base 29: mod_base = OTHER; note = D-LNA
  modified_base 31: mod_base = OTHER; note = D-LNA
  modified_base 33: mod_base = OTHER; note = D-LNA
  modified_base 35: mod_base = OTHER; note = D-LNA

SEQ ID NO: 168 - DNA, 27 bp, synthetic construct
  source 1..27: mol_type = other DNA
  modified_base 2: mod_base = OTHER; note = D-LNA
  modified_base 6: mod_base = OTHER; note = D-LNA
  modified_base 10: mod_base = OTHER; note = D-LNA
  modified_base 14: mod_base = OTHER; note = D-LNA
  modified_base 16: mod_base = OTHER; note = D-LNA
  modified_base 21: mod_base = OTHER; note = D-LNA
tttttttttttttttttgaagctccaa

SEQ ID NO: 169 - DNA, 10 bp, synthetic construct
  source 1..10: mol_type = other DNA
  modified_base 1: mod_base = OTHER; note = fluorescent dye modified nucleotide
  modified_base 10: mod_base = OTHER; note = Black hole quencher 2 attached to a nucleotide sequence
agaactccct

SEQ ID NO: 170 - DNA, 13 bp, synthetic construct
  source 1..13: mol_type = other DNA
  modified_base 1: mod_base = OTHER; note = Black hole quencher 2 attached to a nucleotide sequence
  modified_base 13: mod_base = OTHER; note = spacer modified nucleotide
cgcctcgcagacg

SEQ ID NO: 171 - DNA, 10 bp, synthetic construct
  source 1..10: mol_type = other DNA
  modified_base 1: mod_base = OTHER; note = fluorescent dye modified nucleotide
  modified_base 10: mod_base = OTHER; note = Black hole quencher 2 attached to a nucleotide sequence
ttcaagcctc

SEQ ID NO: 172 - DNA, 10 bp, synthetic construct
  source 1..10: mol_type = other DNA
  modified_base 1: mod_base = OTHER; note = Black hole quencher 2 attached to a nucleotide sequence
  modified_base 10: mod_base = OTHER; note = Spacer modified nucleotide
caagctgtgc

SEQ ID NO: 173 - DNA, 12 bp, synthetic construct
  source 1..12: mol_type = other DNA
  modified_base 1: mod_base = OTHER; note = Black hole quencher 2 attached to a nucleotide sequence
  modified_base 12: mod_base = OTHER; note = Spacer modified nucleotide
tccaagctgtgc

SEQ ID NO: 174 - DNA, 19 bp, synthetic construct
  source 1..19: mol_type = other DNA
  modified_base 1: mod_base = OTHER; note = Black hole quencher 2 attached to a nucleotide sequence
  modified_base 19: mod_base = OTHER; note = Spacer modified nucleotide
cctcgcctcgcagacgaag

SEQ ID NO: 175 - DNA, 15 bp, synthetic construct
  source 1..15: mol_type = other DNA
  modified_base 1: mod_base = OTHER; note = Black hole quencher 2 attached to a nucleotide sequence
  modified_base 15: mod_base = OTHER; note = spacer modified nucleotide
ctcgcctcgcagacg

SEQ ID NO: 176 - DNA, 16 bp, synthetic construct
  source 1..16: mol_type = other DNA
  modified_base 1: mod_base = OTHER; note = Black hole quencher 2 attached to a nucleotide sequence
  modified_base 16: mod_base = OTHER; note = Spacer modified nucleotide
cgcctcgcagacgaag

SEQ ID NO: 177 - DNA, 21 bp, synthetic construct
  source 1..21: mol_type = other DNA
  modified_base 1: mod_base = OTHER; note = Black hole quencher 2 attached to a nucleotide sequence
  modified_base 21: mod_base = OTHER; note = Spacer modified nucleotide
gccttctcatctgccggaccg

SEQ ID NO: 178 - DNA, 10 bp, synthetic construct
  source 1..10: mol_type = other DNA
  modified_base 1: mod_base = OTHER; note = fluorescent dye modified nucleotide
  modified_base 10: mod_base = OTHER; note = Black hole quencher 2 attached to a nucleotide sequence
tctgtgcctt

SEQ ID NO: 179 - DNA, 16 bp, synthetic construct
  source 1..16: mol_type = other DNA
  modified_base 1: mod_base = OTHER; note = Black hole quencher 2 attached to a nucleotide sequence
  modified_base 16: mod_base = OTHER; note = Spacer modified nucleotide
ctcatctgccggaccg

SEQ ID NO: 180 - DNA, 18 bp, synthetic construct
  source 1..18: mol_type = other DNA
  modified_base 1: mod_base = OTHER; note = Black hole quencher 2 attached to a nucleotide sequence
  modified_base 18: mod_base = OTHER; note = Spacer modified nucleotide
ttctcatctgccggaccg

SEQ ID NO: 181 - DNA, 10 bp, synthetic construct
  source 1..10: mol_type = other DNA
  modified_base 1: mod_base = OTHER; note = fluorescent dye modified nucleotide
  modified_base 10: mod_base = OTHER; note = Black hole quencher 2 attached to a nucleotide sequence
cgtctgtgcc